(19) United States
(12) Patent Application Publication
(10) Pub. No.: US 2026/0141987 A1
(43) Pub. Date: May 21, 2026

(54) MULTI-OBJECTIVE OPTIMIZATION FOR MOLECULE DESIGN

(71) Applicants: Genentech, Inc., South San Francisco, CA (US); Hoffmann-La Roche Inc., Little Falls, NJ (US)

(72) Inventors: Natasa TAGASOVSKA, Basel (CH); Ji Won PARK, Foster City, CA (US)

(21) Appl. No.: 19/390,386

(22) Filed: Nov. 14, 2025

Related U.S. Application Data
(63) Continuation of application No. PCT/US2024/029979, filed on May 17, 2024.
(60) Provisional application No. 63/502,658, filed on May 17, 2023.
(60) Provisional application No. 63/515,447, filed on Jul. 25, 2023.

(51) Int. Cl. G16C 20/50; G16C 20/70

(52) U.S. Cl. CPC G16C 20/50; G16C 20/70

(57) ABSTRACT

A plurality of molecule designs may be generated computationally. One or more property computation models may be applied to determine multiple properties of each molecule design. Each property computation model may be trained to approximate a probability distribution of the possible values of a corresponding property. A cumulative distribution function indicator corresponding to an expected multivariate rank may be determined for each molecule design based on the output of the property computation models. The multivariate rank of a molecule design may quantify the probability that none of its properties can be improved without degrading at least one other property. One or more molecule designs may be selected as candidates for wet lab assessment based on the cumulative distribution function indicator of each molecule design. The molecule designs that are selected for wet lab assessment may exhibit incrementally better properties than those from previous design iterations.

### CROSS REFERENCE TO RELATED APPLICATIONS

This application claims priority to U.S. Provisional Application No. 63/502,658, entitled “MULTI-OBJECTIVE ACTIVE LEARNING FOR MOLECULE DESIGN” and filed on May 17, 2023, and U.S. Provisional Application No. 63/515,447, entitled “MULTI-OBJECTIVE ACTIVE LEARNING FOR MOLECULE DESIGN” and filed on Jul. 25, 2023, the disclosures of which are incorporated herein by reference in their entirety.

### TECHNICAL FIELD

The subject matter described herein relates generally to molecular design and more specifically to a multi-objective active learning technique for molecule design.

### INTRODUCTION

A molecule is a group of two more atoms held together by chemical bonds. Molecules form the smallest identifiable unit into which a pure substance can be divided while still retaining the composition and chemical properties of that substance. Various properties of a molecule, including its ability to function as a therapeutic, may be contingent upon the conformation (or three-dimensional structure) of the molecule. One example of a molecule is a small molecule, which is a low-weight compound having a molecular weight between approximately 100 Daltons and 1000 Daltons. Small molecule therapeutics, which modulate biochemical processes to diagnose, treat, and prevent a gamut of illnesses, have been a cornerstone in modern pharmacology due to a number of compelling advantages. For example, small molecule drugs are capable of penetrating cell membranes to reach intracellular targets. Moreover, small molecule drugs are adaptable to a wide variety of therapeutic applications. For instance, a small molecule drug may be formulated as pills and capsules, intravenous or subcutaneous injectables, inhalational medicines, or suppositories. The development of the small molecule drug may further extend to tailoring various pharmacokinetic properties including liberation, absorption, distribution, metabolism, potency, efficacy, phenotypic effects, and excretion.

By contrast, large molecules (also known as biopharmaceuticals, biologicals, or biologics) can range between approximately 3000 Daltons and 150,000 Daltons in molecular weight. Large molecule drugs are often derivatives of natural human proteins, which modulate many essential cellular functions such as enzymatic reactions, transport of molecules, regulation and execution of a number of biological pathways, cell growth, proliferation, nutrient uptake, morphology, motility, intercellular communication, and/or the like. It is common for a single large molecule to have more than 1,300 amino acid residues, which are linked by peptide bonds to form one or more polypeptide. Due to their size and complexity, large molecule drugs are recombinantly produced by engineered cells instead of being chemically synthesized like the majority of small molecule drugs. Moreover, large molecule therapeutics are usually delivered through injection or infusion due to the ineffectiveness of oral administration. The development of a large molecule drug may entail designing one or more sequences of amino acid residues capable of binding to a target (e.g., a protein, a nucleic acid, and/or the like) with sufficient specificity and absent undesirable traits such as immunogenicity, self-association, instability, and/or the like.

### SUMMARY

Systems, methods, and articles of manufacture, including computer program products, are provided for molecule design with multi-objective optimization in which molecule designs are selected as candidates for wet lab evaluation (e.g., synthesis, in vitro measurements, in vivo characterization, and/or the like) based on multiple objectives. In one aspect, there is provided a system for molecule design with multi-objective optimization. The system may include at least one data processor and at least one memory. The at least one memory may store instructions, which causes operations when executed by the at least one data processor. The operations may include: generating a plurality of molecule designs; applying one or more property computation models to determine a plurality of properties exhibited by the plurality of molecule designs, where the one or more property computation models are trained to approximate a probability distribution of each property of the plurality of properties; determining, based at least on an output of the one or more property computation models, a cumulative distribution function (CDF) indicator for each molecule design of the plurality of molecule designs, where the cumulative distribution function (CDF) indicator of a molecule design corresponds to a multivariate rank of the molecule design, and where the multivariate rank of the molecule design quantifies a probability that none of the plurality of properties present in the molecule design can be improved without degrading at least one other property of the plurality of properties; and selecting, based at least on the cumulative distribution function (CDF) indicator of each molecule design, one or more molecule designs from the plurality of molecule designs as candidates for wet lab assessment.

In another aspect, there is provided a method for molecule design with multi-objective optimization. The method may include: generating a plurality of molecule designs; applying one or more property computation models to determine a plurality of properties exhibited by the plurality of molecule designs, where the one or more property computation models are trained to approximate a probability distribution of each property of the plurality of properties; determining, based at least on an output of the one or more property computation models, a cumulative distribution function (CDF) indicator for each molecule design of the plurality of molecule designs, where the cumulative distribution function (CDF) indicator of a molecule design corresponds to a multivariate rank of the molecule design, and where the multivariate rank of the molecule design quantifies a probability that none of the plurality of properties present in the molecule design can be improved without degrading at least one other property of the plurality of properties; and selecting, based at least on the cumulative distribution function (CDF) indicator of each molecule design, one or more molecule designs from the plurality of molecule designs as candidates for wet lab assessment.

In another aspect, there is provided a non-transitory computer program product for molecule design with multi-objective optimization. The non-transitory computer program product may store instructions that cause operations when performed by at least one data processor. The operations may include: generating a plurality of molecule designs; applying one or more property computation models to determine a plurality of properties exhibited by the plurality of molecule designs, where the one or more property computation models are trained to approximate a probability distribution of each property of the plurality of properties; determining, based at least on an output of the one or more property computation models, a cumulative distribution function (CDF) indicator for each molecule design of the plurality of molecule designs, where the cumulative distribution function (CDF) indicator of a molecule design corresponds to a multivariate rank of the molecule design, and where the multivariate rank of the molecule design quantifies a probability that none of the plurality of properties present in the molecule design can be improved without degrading at least one other property of the plurality of properties; and selecting, based at least on the cumulative distribution function (CDF) indicator of each molecule design, one or more molecule designs from the plurality of molecule designs as candidates for wet lab assessment.

In some variations of the methods, systems, and non-transitory computer readable media, one or more of the following features can optionally be included in any feasible combination.

In some variations, the one or more property computation models may be trained to approximate a first probability distribution of a first plurality of possible values of a first property of the plurality of properties. The one or more property computation models may be further trained to approximate a second probability distribution of a second plurality of possible values of a second property of the plurality of properties.

In some variations, the output of the one or more property computation models may include a plurality of predictive samples from the first probability distribution and the second probability distribution. Each predictive sample may include a first value of the first property and a second value of the second property present in the molecule design.

In some variations, the cumulative distribution function (CDF) indicator of each molecule design may be determined by at least determining, based at least on the output of the one or more property computation models, a marginal distribution of each property of the plurality of properties, determining, based at least on the output of the one or more property computation models, one or more copulas describing an inter-correlation between the plurality of properties, and determining, based at least on the marginal distribution of each property and the one or more copulas, the cumulative distribution function (CDF) indicator of each molecule design.

In some variations, the cumulative distribution function (CDF) indicator of each molecule design may be further determined by at least determining a third marginal distribution of a third property of the plurality of properties, and determining a second copula coupling the third marginal distribution and at least one of the first marginal distribution and the second marginal distribution.

In some variations, the first copula and the second copula may be bivariate copulas forming a vine.

In some variations, the vine may be determined to exhibit a hierarchical structure corresponding to a partial ordering in which the first property is prioritized over the second property and/or the third property.

In some variations, the cumulative distribution function (CDF) indicator of each molecule design may be determined by at least performing a pairwise factorization of a multivariate joint distribution corresponding to the plurality of properties to determine one or more pairwise groupings of the plurality of properties, where each pairwise grouping of the plurality of properties corresponds to a bivariate joint distribution, and determining a bivariate copula coupling each pairwise grouping of the plurality of properties.

In some variations, the cumulative distribution function (CDF) indicator of each molecule design may be determined by at least determining, based at least on a tail behavior of the bivariate joint distribution, a type of the bivariate copula coupling each pairwise grouping of the plurality of properties.

In some variations, the type of bivariate copula may be one of a Clayton copula, a Gumbel copula, or a Gaussian copula.

In some variations, the cumulative distribution function (CDF) indicator of each molecule design may be determined by at least determining, based at least on the measurement set, a mean and covariance of the plurality of properties, determining, based at least on the mean and covariance of the plurality of properties, a multivariate Gaussian distribution of a plurality of possible values of the plurality of properties, and determining, based at least on the multivariate Gaussian distribution, the cumulative distribution function (CDF) indicator of each molecule design.

In some variations, the cumulative distribution function (CDF) indicator of each molecule design may be determined by at least determining, based at least on the measurement set, an empirical cumulative distribution function, where the empirical cumulative distribution function comprises a step function that increases by

$$\frac{1}{n}$$

for each of an n quantity of datapoints in the measurement set, and where the empirical cumulative distribution function outputs, for any specified value of the plurality of properties, a value corresponding to a fraction of measurements in the measurement set that are less than or equal to the specified value, and determining, based at least on the empirical cumulative distribution function, the cumulative distribution function (CDF) indicator of each molecule design.

In some variations, the cumulative distribution function (CDF) indicator of each molecule design may be determined by at least performing a kernel density estimation (KDE) to estimate a multivariate joint distribution corresponding to the plurality of properties, and determining, based at least on an estimate of the multivariate joint distribution, the cumulative distribution function (CDF) indicator of each molecule design.

In some variations, the cumulative distribution function (CDF) indicator of each molecule design may be an expected cumulative distribution function (CDF) indicator whose value is determined to account for an uncertainty in the output of the one or more property computation models.

In some variations, the one or more molecule designs may be selected as candidates for wet lab assessment based at least on the one or more molecule designs having a better cumulative distribution function (CDF) indicator than one or more molecule designs generated during a previous iteration of multi-objective Bayesian optimization.

In some variations, a first molecule design may be selected as a candidate for wet lab assessment based at least on a first cumulative distribution function (CDF) indicator of the first molecule design satisfying one or more thresholds. A second molecule design may be excluded from being a candidate for wet lab assessment based at least on a second cumulative distribution function (CDF) indicator of the second molecule design failing to satisfy the one or more thresholds.

In some variations, a first molecule design may be selected instead of a second molecule design as a candidate for wet lab assessment based at least on a first cumulative distribution function (CDF) indicator of the first molecule design being better than a second cumulative distribution function (CDF) indicator of the second molecule design.

In some variations, a threshold quantity of molecule designs having a best cumulative distribution function (CDF) indicator may be selected based at least on the cumulative distribution function (CDF) indicator of each molecule design as candidates for wet lab assessment.

In some variations, a measurement set associated with a plurality of prior molecule designs may be received. The measurement set may include, for each prior molecule design, one or more measurements of a plurality of properties exhibited by each prior molecule design. One or more property computation models may be trained, based at least on the measurement set, to approximate the probability distribution of each property of the plurality of properties

In some variations, one or more additional measurements for the plurality of properties may be received for the one or more molecule designs selected as candidates for wet lab assessment. The one or more property computation models may be retrained based at least on the one or more additional measurements. The one or more retrained property computation models may be applied to determine the plurality of properties exhibited by one or more subsequent molecule designs.

In some variations, the retraining of the one or more property computation models may include updating, based at least on the one or more additional measurements, the probability distribution of each property of the plurality of properties being approximated by the one or more property computation models.

In some variations, the plurality of prior molecule designs may be generated during a previous iteration of multi-objective Bayesian optimization (MOBO). The plurality of molecule designs may be generated during a current design iteration of multi-objective Bayesian optimization (MOBO). The one or more subsequent molecule designs may be generated during a subsequent iteration of multi-objective Bayesian optimization (MOBO).

In some variations, the one or more property computation models may include at least one ensemble of property computation models in which multiple property computation models are trained to determine a single property of the plurality of properties.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the design of large molecules such as protein molecules, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a molecule design system, in accordance with some example embodiments;

FIG. 2A depicts a flowchart illustrating an example of a process for molecule design with multi-objective optimization, in accordance with some example embodiments;

FIG. 2B depicts a flowchart illustrating another example of a process for molecule design with multi-objective optimization, in accordance with some example embodiments;

FIG. 2C depicts a flowchart illustrating another example of a process for molecule design with multi-objective optimization, in accordance with some example embodiments;

FIG. 3A depicts a flowchart illustrating an example of a process for determining the property of a molecule design, in accordance with some example embodiments;

FIG. 3B depicts a flowchart illustrating another example of a process for determining the property of a molecule design, in accordance with some example embodiments;

FIG. 4A depicts graphs illustrating a comparison of different utility metrics including hypervolume, multivariate ranks, and cumulative distribution function (CDF) indicators, in accordance with some example embodiments;

FIG. 4B depicts a graph illustrating another comparison of different utility metrics including hypervolume, multivariate ranks, and cumulative distribution function (CDF) indicators, in accordance with some example embodiments;

FIG. 4C depicts graphs illustrating a comparison of the level lines of the cumulative distribution function (CDF) and probability distribution function (PDF) from kernel density estimation (KDE), in accordance with some example embodiments;

FIG. 5A depicts a schematic diagram illustrating an example of a multivariate joint distribution being estimated using marginal distributions and a copula joining the marginal distributions, in accordance with some example embodiments;

FIG. 5B depicts a schematic diagram illustrating an example of vine copula decomposition, in accordance with some example embodiments;

FIG. 5C is a schematic diagram illustrating the use of copulas in the context of optimizing multiple objective in tasks with sparse data, in accordance with some example embodiments;

FIG. 6 depicts a graphs illustrating a comparison of the rescaling of different utility metrics including cumulative distribution function (CDF) indicator and hypervolume, in accordance with some example embodiments;

FIG. 7 depicts graphs illustrating changes in the values of hypervolume (HV) and cumulative distribution function (CDF) indicator of molecule designs obtained over simulated iterations of Bayesian optimization for the Branin-Currin test function and the DTLZ problem collection, in accordance with some example embodiments;

FIG. 8 depicts a graph illustrating a comparison of the time complexity for different acquisition functions including improvement-based acquisition functions and multivariate ranking based acquisition functions, in accordance with some example embodiments;

FIG. 9A depicts two examples of molecules exhibiting desirable properties, in accordance with some example embodiments;

FIG. 9B depicts an example of a molecule exhibiting undesirable properties, in accordance with some example embodiments; and

FIG. 10 depicts a block diagram illustrating an example of a computing system, in accordance with some example embodiments.

When practical, similar reference numbers denote similar structures, features, or elements.

### DETAILED DESCRIPTION

A molecule may be designed to exhibit multiple desirable properties including, in the case of therapeutics, drug-like properties such as affinity, specificity, biological activity, developability, and/or the like. In some cases, whether a molecule exhibits certain desirable properties may be contingent on the molecule being able to adopt a corresponding conformation (or three-dimensional structure). For example, the binding affinity between a drug molecule and a target molecule (e.g., a protein, a nucleic acid, and/or the like) may depend on the ability of the drug molecule to adopt a conformation (or three-dimensional structure) that is complementary to that of the target molecule. As such, in some cases, designing a molecule may include determining the composition as well as the conformation (or three-dimensional structure) of the molecule. For instance, in the case of small molecules (e.g., low-weight compounds having a molecular weight between approximately 100-1000 Daltons), the design process may include scoring and ranking different molecules selected from at least a portion of the molecular space (or chemical space) populated by every possible chemical compound (e.g., every possible combination of atoms of two or more chemical elements). For larger molecules, such as protein molecules whose primary structure is a linear sequence of amino acid residues linked to from one or more polypeptide chains, the design process may include determining the identities of the amino acid residues in each polypeptide chain and the conformation (or three-dimensional structure) assumed by the folding of the polypeptide chains.

Despite the time and cost efficiency achieved through the adoption of in silico design tools, computational molecule design remains an intractable task when tackled with conventional brute force approaches that generate molecule designs through indiscriminate searches of the vast combinatorial space of possible molecular compositions and conformations. This is because the near infinite quantity of possible molecular compositions and conformations is overwhelming for even state-of-the-art computational resources. For example, in the case of small molecules, the molecular space (or chemical space) is estimated to contain 10⁶⁰ possible chemical compounds and scales exponentially with molecule size (e.g., the number of constituent atoms), meaning that state-of-the-art computational resources can support the exploration of only a small fraction of the molecular space (e.g., small regions of the molecular space selected based on prior domain knowledge). The size of the combinatorial space is magnitudes larger for larger molecules and biologics. For instance, for protein molecules containing an N quantity of amino acid residues, approximately 20^{N} possible protein sequences exist when each of the N quantity of amino acid residues is one of the twenty canonical amino acid residues. Each one of the aforementioned 20^{N} possible protein sequences is further capable of adopting an exponential number of conformations (or three-dimensional structures). Even in cases where each one of the N quantity of amino acid residues in a possible protein sequence is limited to assuming one of an M quantity of discrete geometric states (e.g., rotamers), every one of the aforementioned 20^{N} possible protein sequences may still adopt M^{N} possible conformations (or three-dimensional structures).

In some example embodiments of the present disclosure, a molecule design engine may improve upon the current practice of an indiscriminate exploration of the vast combinatorial space of possible molecular compositions and conformations (or three-dimensional structures). Rather, a molecule design engine of the present disclosure may generate one or more molecule designs by sampling, in a principled manner, a data distribution of molecules exhibiting one or more desirable properties. For example, in some cases, the molecule design engine may generate the one or more molecule designs by sampling a data distribution of protein molecules or non-protein molecules such as small molecules, ions, nucleic acids, polysaccharides, glycolipids, and/or the like. In the case of drug design, the data distribution may be populated by molecules exhibiting drug-like properties such as affinity, specificity, biological activity, developability, and/or the like. In some cases, the molecule design engine may include a molecule design computation model trained to approximate the data distribution such that the one or more molecule designs may be generated by sampling the one or more molecule designs from regions in the data distribution more likely to be populated by molecules exhibiting the one or more desirable properties. For instance, in some cases, the molecule design computation model may be trained to approximate the data distribution based on a training dataset of known molecules exhibiting the one or more desirable properties.

In some example embodiments, training the molecule design computation model to approximate the data distribution may include adjusting one or more parameters (e.g., weights, biases, and/or the like) of the molecule design computation model to increase (or maximize) the similarity between the molecule designs output by the molecule design computation model and the known molecules in the training dataset. In some cases, doing so may also include determining a function (e.g., a score function, an energy function, and/or the like) whose output (e.g., score, energy value, and/or the like) differentiates between higher density regions of the data distribution more likely to be populated by molecules exhibiting the one or more desirable properties and lower density regions of the data distribution less likely to be populated by molecules exhibiting the one or more desirable properties. Accordingly, once trained, the molecule design computation model may be applied to generate one or more molecule designs. For example, the one or more molecules designs may be generated through one or more iterations of gradient-based Markov Chain Monte Carlo (MCMC) sampling (e.g., Markov Chain Monte Carlo (MCMC) sampling with Langevin dynamics and/or the like). In some cases, each iteration of gradient-based Markov Chain Monte Carlo (MCMC) sampling may include drawing, from the data distribution, a sample (or molecule) by applying the molecule design computation model to modify an initial molecule design, for example, of a known molecule or a noise molecule. The sampling may be performed based on the function (e.g., score function, energy function, and/or the like) such that each successive sampling iteration includes drawing a sample (or molecule) from an incrementally higher density region of the data distribution.

While the molecule design computation model may accelerate the generation of molecule designs, the limited availability and exorbitant cost of laboratory resources still impose a significant bottleneck on the rate at which the molecule designs generated by the molecule design computation model can undergo wet lab assessment. For example, in a typical drug development pipeline, a molecule design must be validated in vitro and undergo multiple rounds of optimization before that molecule design can proceed to preclinical development and clinical trials where the performance of the molecule is tested in vivo. Even though the molecule design computation model may be capable of generating a large number of molecule designs (e.g., in the order of millions of molecule designs) quickly and with comparatively little cost, practical limitations when it comes to wet lab resources will preclude wet lab assessment of every molecule design. Instead, only a subset of the molecule designs generated by the molecule design computation model may be selected for wet lab assessment including, for example, in vitro measurements, in vivo characterization, and/or the like.

In some example embodiments, molecule designs may be generated computationally and assessed in the web lab over multiple successive design iterations, with each design iteration generating one or more molecule designs with better molecule properties than those from previous design iterations. However, indiscriminately selecting molecule designs for wet lab assessment may increase the risk that those with poor molecular properties, such as suboptimal pharmacological and physiochemical properties, advance to and fail during subsequent stages of the drug development pipeline while better molecule designs are overlooked. Accordingly, instead of an indiscriminate selection of molecule designs, the ones that are selected for wet lab assessment during a current design iteration should exhibit better molecular properties than those from previous design iterations. As described in more detail below, in some example embodiments, a selection engine may perform multi-objective optimization, such as multi-objective Bayesian optimization (MOBO), across a set of mixed variable molecular properties when identifying which molecule designs generated by the molecule design computation model are selected for further wet lab assessment (e.g., in vitro measurements, in vivo characterization, and/or the like). In this context, each objective may correspond to a molecular property (e.g., a drug-like property such as affinity, specificity, biological activity, developability, and/or the like) that is being optimized over multiple rounds of computational molecule design and wet lab assessments. Accordingly, in some cases, the selection engine may perform multi-objective optimization by exploring an objective space occupied by various molecule designs, each of which having a different combination of objectives (or molecular properties). Doing so in a principled manner may increase the likelihood that better molecule designs, such as those exhibiting better molecular properties than those from previous design iterations, are selected for wet lab assessment (e.g., in vitro measurements, in vivo characterization, and/or the like).

In some example embodiments, the selection engine may perform multi-objective Bayesian optimization (MOBO) in order to explore the aforementioned objective space in a principled manner. For example, the selection engine may apply one or more property computation models trained to approximate a first probability distribution of the possible values of a first property as well as a second probability distribution of the possible values of a second property. That is, the one or more property computation models may be applied to determine, for each molecule design generated by the molecule design engine, one or more predictive samples from the first probability distribution and the second probability distribution. Each predictive sample in this case may include a first value of the first property and a second value of the second property present in the molecule design. In this context, the one or more property computation models may serve as an in silico surrogate for in vitro and/or in vivo evaluations which, as noted, are too resource intensive to apply to every molecule design generated by the molecule design computation model. In some cases, the multi-objective Bayesian optimization (MOBO) may include a tradeoff between an exploration and exploitation of the objective space. For instance, in some cases, the multi-objective Bayesian optimization (MOBO) may include sampling, from the objective space, a selection of molecule designs that balances the exploration of highly uncertain molecule designs (e.g., molecule designs whose molecular properties are highly uncertain) with the exploitation of those more likely to increase or maximize the objectives (e.g., molecule designs likely to exhibit better molecular properties).

In some example embodiments, the one or more property computation models may be implemented as one or more probabilistic surrogate models in order to account of the uncertainty associated with the inferring the molecular properties of each molecule design. Because the one or more property computation models are trained on wet lab measurements of the individual molecular properties, this uncertainty may be attributable, at least in part, to the noise that may be present in those wet lab measurements. As such, in some cases, each property computation model may be implemented as a probabilistic surrogate model trained to approximate a probability distribution of the possible values of a corresponding molecular property exhibited by that molecule design. Moreover, in some cases, the one or more property computation models may output, based at least on the probability distribution, one or more predictive samples for each molecule design generated by the molecule design engine. For instance, in cases where the molecule designs are being optimized for two objectives (or molecular properties), each predictive sample may include a first value of a first property exhibited by the first molecule design as well as a second value of a second property exhibited by the first molecule design having the first value of the first property.

In some example embodiments, the selection engine may perform multi-objective Bayesian optimization (MOBO) and explore the objective space in a principled manner in order to identify, based on the predictive samples output by the one or more property computation models, one or more molecule designs that meet certain criteria with respect to multiple molecular properties (e.g., drug-like properties). It should be appreciated that these criteria do not necessarily require a molecule design to have the best value for every molecular property of interest (e.g., highest expression level and highest binding affinity) at least because such a molecule design may not exist at all. Instead, at least some of the molecular properties of interest may be competitive in that the enhancement of one molecular property may come at the impairment of one or more others. As such, the selection engine may perform multi-objective Bayesian optimization (MOBO) to identify molecule designs in which no properties can be further improved without degrading at least one other property. In some cases, such a molecule design may be called a Pareto-optimal solution or a nondominated solution. A set of Pareto-optimal solutions, which may contain one or more such molecule designs, may form a Pareto frontier (or Pareto front). Accordingly, in some cases, the selection engine may perform multi-objective Bayesian optimization (MOBO) in order to determine, during each design iteration, a set of Pareto-optimal solutions as candidates for wet lab assessment.

In some example embodiments, each Pareto-optimal solution may be identified by the selection engine applying an acquisition function. In some cases, the acquisition function may output, for each molecule design generated by molecule design computation model, a utility metric indicative of the expected utility of the molecule design. For example, in the context of drug design, the utility metric of a molecule design may correspond to the probability of the molecule design being one of the Pareto-optimal solutions populating the Pareto frontier. In some cases, the utility metric of the molecule design may correspond to the distance (or proximity) between the molecule design and the Pareto frontier, meaning that the molecule design may be considered a Pareto-optimal solution on the Pareto frontier if utility metric of the molecule design satisfies one or more thresholds. As noted, in some cases, a molecule design that qualifies as a Pareto-optimal solution whose utility metric satisfies one or more thresholds may be identified as a candidate for wet lab assessment (e.g., in vitro measurements, in vivo characterization, and/or the like). Furthermore, in some cases, at least some of the molecule designs included in the set of Pareto-optimal solutions identified for a current design iteration may become baseline molecule designs for one or more subsequent design iterations and serve as a basis for identifying the next set of Pareto-optimal solutions.

In some example embodiments, instead of the conventional acquisition functions used for multi-objective Bayesian optimization (MOBO), the selection engine may apply a multivariate ranking based acquisition function. Doing so may reduce the time complexity of identifying Pareto-optimal solutions across multiple, oftentimes competing objectives (or molecular properties of interest) as conventional acquisition functions scale poorly to accommodate the larger quantities of objectives (or molecular properties of interest) typically found in the context of drug design. For example, conventional acquisition functions are sensitive to even the non-informative transformations of individual objectives, such as rescaling one objective relative to another or monotonic transformation of a single objective. Such transformation are often performed to standardize across different units of measurement, which are common between different objectives (or molecular properties of interest) in drug design.

An improvement-based acquisition function is one example of a conventional acquisition function with poor time complexity. In cases where the selection engine applies an improvement-based acquisition function, for example, the utility metric resulting therefrom may correspond to a difference between a first hypervolume of a first polytope bounded by the combination of molecular properties associated with the molecule design and a second hypervolume of a second polytope bounded by the combination of molecular properties associated with the baseline molecule designs. However, the time complexity of computing hypervolume (HV), which requires determining the volume of irregularly shaped polytopes, scales in a super-polynomial manner with the number of objectives (or molecular properties of interest). As such, despite the efficiency of some state-of-the art techniques for computing hypervolume such as box decomposition, applying an improvement-based acquisition function to identify Pareto-optimal solutions remains slow for even a relatively small number of objectives (or molecular properties of interest).

Another example of a conventional acquisition function is an entropy-based acquisition function that focuses on increasing (or maximizing)th information gain from subsequent observations (e.g., next set of Pareto-optimal solutions). However, while improvement-based acquisition functions require computing the volume of an irregularly-shaped volume, entropy-based acquisition functions require computing high-dimensional definite integrals. For example, in some cases, the utility metric for an entropy-based acquisition function may be determined by computing the high-dimensional definite integral of an M-dimensional multivariate Gaussian distribution, where M is the number of objectives (or molecular properties of interest). Even with more efficient techniques like box decomposition, the high-dimensional definite integral of an M-dimensional multivariate Gaussian distribution is still more costly to evaluate than even hypervolume (HV).

In some example embodiments, the time complexity of identifying Pareto-optimal solutions across multiple objectives (or molecular properties of interest), particularly a large quantities of objectives in the case of drug design, may be reduced by the selection engine applying a multivariate ranking based acquisition function. For example, in some cases, the multivariate ranking based acquisition function may output a utility metric corresponding to the expected multivariate rank of each molecule design generated by the molecule design computation model. In some cases, the expected multivariate rank of a molecule design may be a ranking of the molecule design based on multiple properties present in the molecule design. In the context of antibody design, for example, the multivariate rank of a molecule design may be a ranking of the molecule design based on its expression level, affinity, and developability traits. The multivariate ranking based function may output the expected multivariate rank of each molecule design in order to account for the uncertainty that may be present in the output of the probabilistic surrogate models. For, in some cases, the multivariate rank of a molecule design may correspond to the probability of the molecule design being one of the Pareto-optimal solutions populating the Pareto frontier. As such, in some cases, the expected multivariate rank of a molecule design may be indicative of its distance (or proximity) to the true Pareto frontier (or Pareto front). Moreover, in some cases, the multivariate rank of a molecule design may quantity the quality of the molecule design as a Pareto-optimal solution, meaning that a molecule design that is more proximate to the Pareto frontier may be associated with a different multivariate rank than a molecule design that is more distant from the Pareto frontier.

However, unlike ranking multiple molecule designs based on a single objective (or molecular property of interest), determining the multivariate rank of individual molecule designs, which is tantamount to ranking in high dimensions based on multiple objectives (or molecular properties of interest), is a still nontrivial task. This is because there is no natural ordering (or ranking) in Euclidean space when the number M of objectives (or molecular properties of interest) is greater than one (e.g., M≥2). Accordingly, in some example embodiments, the selection engine may determine, as the utility metric of each molecule design generated by the molecule design computation model, a cumulative distribution function (CDF) indicator consistent with the multivariate rank of the molecule design. Moreover, in some cases, the Pareto-optimal solutions for each design iteration may be identified based on the cumulative distribution function (CDF) indicator of each molecule design generated by the molecule design computation model. For example, in some cases, molecule designs that are Pareto-optimal solutions on the Pareto frontier may be associated with the same (or tied) multivariate ranks, meaning that corresponding cumulative distribution function (CDF) indicators are also the same (or tied). The cumulative design function (CDF) indicator of a molecule design may be estimated in a variety of different ways including, for example, using copulas, empirical cumulative distribution function (CDF), kernel density estimation (KDE), multivariate Gaussian cumulative distribution function (CDF), and/or the like. As described in more details below, when estimated using copulas, the cumulative distribution function (CDF) indicator of each molecule design may be a particularly robust utility metric with greater scalability than hypervolume (HV).

In some example embodiments, the cumulative distribution function (CDF) indicator of a molecule design may quantify the quality of the molecule design as a Pareto-optimal solution by at least indicating a probability of the molecule design having a greater function value than other molecule designs. Where there is a single objective (or molecular property of interest), the cumulative distribution function (CDF) indicator of the molecule design may represent the probability that the value of molecular property exhibited by the molecule design satisfies one or more threshold values. Where there are two or more objectives (or molecular properties of interest), the cumulative distribution function (CDF) indicator of the molecule design becomes a multivariate joint distribution corresponding to the maximum multivariate rank of the molecule design. As noted, ranking the molecule design in high dimensions is a nontrivial task at least because computing the multivariate joint distribution is a computationally challenging task that includes, in some cases, estimating the multivariate density function before computing the integral thereof. Accordingly, in some cases, the selection engine may determine the cumulative distribution function (CDF) indicator for a molecule design by at least decomposing the corresponding multivariate joint distribution into two or more constituent marginal distributions and a coupling function called a copula. For example, as described in more details below, the selection engine may decompose the multivariate joint distribution into one or more bivariate joint distributions, each of which including the marginal distributions of two or more paired groupings of objectives (or molecular properties of interest). Furthermore, the multivariate joint distribution may be decomposed into one or more bivariate copulas, each of which coupling a single paired grouping of objectives (or molecular properties of interest). These bivariate copulas may form a type of dependence model called a vine copula. In some cases, additional computational efficiency may be achieved by truncating one or more copulas. Doing so may remove higher order dependencies between some objectives (or molecular properties of interest) that may be sufficiently trivial to be ignored when determining the cumulative distribution function (CDF) indicator of each molecule design. However, it should be appreciated that some copulas may be preserved during truncation in order to preserve the dependency between some objectives (or molecular properties of interest) including, for example, a partial ordering in which some objectives (or molecular properties of interest) are prioritized over others.

In some example embodiments, the selection engine may determine the cumulative distribution function (CDF) indicator of a molecule deign by at least estimating a copula (e.g., bivariate copula) coupling together the marginal distributions of each constituent objective (or molecular property of interest). For example, in cases where the molecule design is being optimized for a first property and a second property, the cumulative distribution function (CDF) indicator of the molecule design may be determined by estimating a copula coupling together a first marginal distribution of the possible values of the first property and a second marginal distribution of the possible values of the second property. In this regard, the copula may describe a dependence structure (e.g., a copula matrix) specifying the inter-correlation between the possible values of the first property and the possible values of the second property. It should be appreciated that the marginal distribution of the possible values of the first property is the probability distribution of those values independent of the possible values of the second property. Meanwhile, the marginal distribution of the possible values of the second property is the probability distribution of these values independent of the possible values of the first property. In instance where the molecule design is being further optimized for a third property, the selection engine may determine the cumulative distribution function (CDF) indicator by at least determining a collection of bivariate copulas, each of which describing the inter-correlation between a pairwise grouping of the first property, the second property, and the third property. The computational complexity of using copulas to estimate the cumulative distribution function (CDF) indicator of the molecule design may be lower than that of determining the underlying multivariate joint distribution at least because each marginal distribution and the corresponding copulas may be estimated separately.

In some example embodiments, the selection engine may determine the collection of bivariate copulas by performing pairwise factorization in which the objectives (or molecular properties of interest) are grouped in pairs. In some cases, the vine copulas may form cascades of bivariate copula blocks. The resulting vine structure may include a hierarchy of nested trees called a vine, which enables the enforcement of dependencies between different objectives (or molecular properties of interest). For example, in cases where a molecule design is being optimized for a first property as well as a second property, a partial ordering of the first property and the second property may require the first property of the molecule design to satisfy a first criteria before the molecule design is assessed to determine whether the second property of the molecule design satisfies a second criteria. In the context of antibody design, this partial ordering may reflect an experimental and/or biological dependency in which the molecule design is required to reach a certain expression level before a sufficient quantity of the molecule design can be synthesized and assayed for other properties such as binding affinity to the target antigen. Accordingly, in some cases, the pairwise factorization may be performed to group objectives (or molecular properties of interest) such that the hierarchy of the resulting vine structure includes two or more conditional copulas encoding the partial ordering in which, for example, the first property is prioritized over the second property.

In some example embodiments, the selection engine may apply an active learning approach in which one or more of the molecule designs selected for wet lab assessment during a current design iteration become the baseline molecule designs during subsequent design iterations. For example, a first molecule design selected for wet lab assessment during the current design iteration may become one of the baseline molecule designs during a subsequent design around. Accordingly, in some cases, the selection engine may select a second molecule design generated by the molecule design computation model during the subsequent design iteration based at least on the utility metric of the second molecule design being an improvement over the utility metrics of the baseline molecule designs and, in some cases, those of the other molecule designs generated for that design iteration. The values of properties used to determine the utility metric of the first molecule design may be determined based on the output of the one or more property computation models. Alternatively and/or additionally, the values of each property present in the first molecule may be determined based on one or more in vitro measurements or in vivo characterizations associated with the first molecule design. In the latter case, to reduce (or minimize) the effects of this noise on the utility metrics computed therefrom, the values of each property present in the first molecule design may be determined based on the output of the one or more property computation models after the one or more property computation models have been updated, for example, by being retrained based on the one or more in vitro measurements or in vivo characterizations associated with the first molecule design.

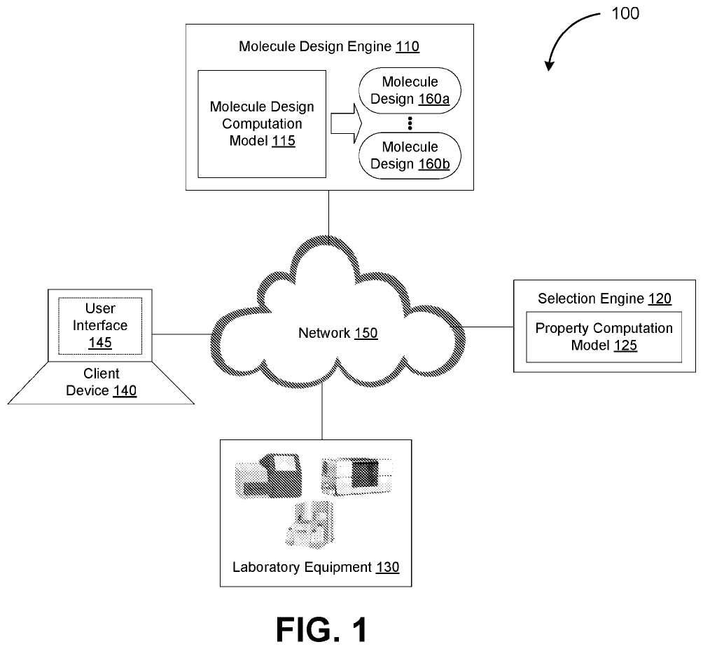

FIG. 1 depicts a system diagram illustrating an example of a molecule design system 100, in accordance with some example embodiments. Referring to FIG. 1 the molecule design system 110 may include a molecule design engine 110, a selection engine 120, one or more wet lab equipment 130, and a client device 140. As shown in FIG. 1, the molecule design engine 110, the selection engine 120, the one or more laboratory equipment 130, and the client device 140 may be communicatively coupled via a network 150. The one or more laboratory equipment 130 may include any wet lab and dry lab equipment capable of performing in vitro measurements and/or in vivo characterizations. Examples of the one or more laboratory equipment 130 may include sequencers, mass spectrometers, centrifuges, and/or the like. The client device 140 may be a processor-based device including, for example, a workstation, a desktop computer, a laptop computer, a smartphone, a tablet computer, a wearable apparatus, and/or the like. The network 150 may be a wired network and/or a wireless network including, for example, a local area network (LAN), a virtual local area network (VLAN), a wide area network (WAN), a public land mobile network (PLMN), the Internet, and/or the like.

Referring again to FIG. 1, the molecule design engine 110 may apply a molecule design computational model 115 to generate multiple molecule designs including, for example, a first molecule design 160a, a second molecule design 160b, and/or the like. For example, in some cases, the first molecule design 160a and the second molecule design 160b may correspond to protein molecules or non-protein molecules such as such as small molecules, ions, nucleic acids, polysaccharides, glycolipids, and/or the like. Moreover, in some cases, the molecule design computational model 115 may be a machine learning model trained to approximate a data distribution of molecules exhibiting one or more desirable properties (e.g., drug-like properties such as affinity, specificity, biological activity, developability, and/or the like). In some cases, the molecule design computation model 115 may be trained by adjusting one or more parameters of the molecule design computation model 115 to increase (or maximize) a similarity between the molecule designs output by the molecule design computation model 115 and known molecules in a training dataset (e.g., known molecules exhibiting the one or more desirable properties). Doing so may include determining a function (e.g., a score function, an energy function, and/or the like) whose parameters correspond to those of the molecule design computation model 115 and whose output (e.g., score, energy value, and/or the like) differentiates between different density regions of the data distribution. In some cases, higher density regions of the data distribution may be more likely to be populated by molecules exhibiting the one or more desirable properties than lower density regions of the data distribution. Accordingly, once trained, the molecule design computation model 115 may sampling from the data distribution in order to generate one or more molecule designs including, for example, the first molecule design 160a, the second molecule design 160, and/or the like. For instance, in some cases, each molecule design, such as the first molecule design 160a and the second molecule design 160b, may be generated through one or more iterations of the gradient-based Markov Chain Monte Carlo (MCMC) sampling (e.g., Langevin Markov Chain Monte Carlo (MCMC) sampling and/or the like). The sampling may be guided by the output (e.g., score, energy value, and/or the like) of the function (e.g., score function, energy function, and/or the like) such that each iteration of the gradient-based Markov Chain Monte Carlo (MCM) sampling includes drawing a sample (or molecule) from an incrementally higher density region of the data distribution.

In some example embodiments, the molecule design engine 110 may be capable of generating a large quantity of molecule designs but not every molecule design generated by the molecule design engine 110 may undergo wet lab assessments such as in vitro measurements, in vivo characterization, and/or the like. In some cases, practical limitations, including the limited availability and exorbitant cost of laboratory resources, may prevent at least some molecule designs generated by the molecule design engine 110 from undergoing wet lab assessment. However, the molecule designs that are selected for wet lab assessment should exhibit satisfactory properties including, in some cases, a superior combination of properties (e.g., drug-like properties) than those from previous design iterations that have already underwent wet lab assessment. Accordingly, in some cases, the selection engine 120 may perform one or more iterations of active learning in order to identify, for synthesis and testing by the one or more laboratory equipment 130, one or more molecule designs that meet specific criteria with respect to one or more properties (e.g., drug-like properties). In some cases, the one or more criteria associated with a property (e.g., drug-like property) may include the value of the property satisfying one or more thresholds, falling within one or more intervals of values, being a member of a set, and/or the like. For example, in the case of antibody design, the selection engine 120 may perform one or more iterations of active learning in order to identify one or more molecule designs that exhibit a sufficient expression level as well as adequate binding affinity towards a target antigen. Furthermore, in some cases, the selection engine 120 may perform the one or more iterations of active learning in order to identify one or more molecule designs that, in addition to having a sufficient expression level and adequate binding affinity, also exhibit certain developability traits such as specificity, thermostability, and/or the like.

In some example embodiments, the selection engine 120 may identify a molecule design for wet lab assessment by at least determining a utility metric indicative of the expected utility of the molecule design. In some cases, the utility metric of the molecule design may correspond to the probability of the molecule design being a Pareto-optimal solution exhibiting a better combination of properties (e.g., drug-like properties) than one or more baseline molecule designs. In some cases, the molecule design being a Pareto-optimal solution may mean that the none of the properties of the molecule design may be further improved without degrading at least one other property of the molecule design. For example, in instances where the selection engine 120 is optimizing a first property and a second property, a molecule design that is a Pareto-optimal solution may exhibit, for a first property, a first value that cannot be further improved without degrading a second value of the second property present in the molecule design. In some cases, the design engine 110 may determine the utility metric of the molecule design by at least applying an acquisition function. However, in order to avoid the excessive computational complexity of a conventional acquisition function (e.g., improvement-based acquisition function, entropy-based acquisition function, and/or the like) or a naïve application of a multivariate ranking based acquisition function, the selection engine 120 may determine, as the utility metric of the molecule design, a cumulative distribution function (CDF) indicator corresponding to a multivariate rank of the molecule design. As described in more details below, in some cases, the selection engine 120 may apply a cumulative distribution function (CDF) acquisition function to determine, for each molecule design generated by the molecule design engine 110, a cumulative distribution function (CDF) indicator corresponding to an expected multivariate rank of the molecule design. In some cases, the selection engine 120 may determine the cumulative distribution function (CDF) acquisition function to determine, based at least on the output of one or more property computation models 125, the expected multivariate rank of the molecule design. In some cases, the cumulative distribution function (CDF) acquisition function may determine the expected multivariate rank of each molecule design to account for the uncertainty that may be present in the output of the one or more property computation models 125.

In some example embodiments, the selection engine 120 may estimate the cumulative distribution function (CDF) indicator of a molecule design in a variety of different ways. For example, in some cases, the selection engine 120 may use copulas (e.g., bivariate copulas) to estimate the cumulative distribution function (CDF) indicator of the molecule design. Accordingly, in some cases, the cumulative distribution function (CDF) indicator of the molecule design may be determined by at least estimating the marginal distribution of each objective being optimized (or molecular property of interest) and one or more copulas (e.g., bivariate copulas) describing the inter-correlation between different objectives. Alternatively, the selection engine 120 may use other estimators, such as a multivariate Gaussian cumulative distribution function (CDF), an empirical cumulative distribution function (CDF), and kernel density estimation (KDE), to determine the cumulative distribution function (CDF) indicator of each molecule design.

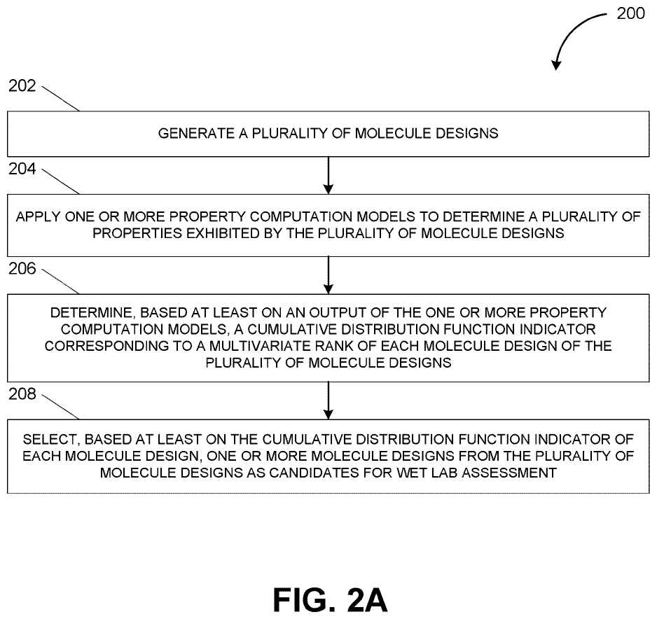

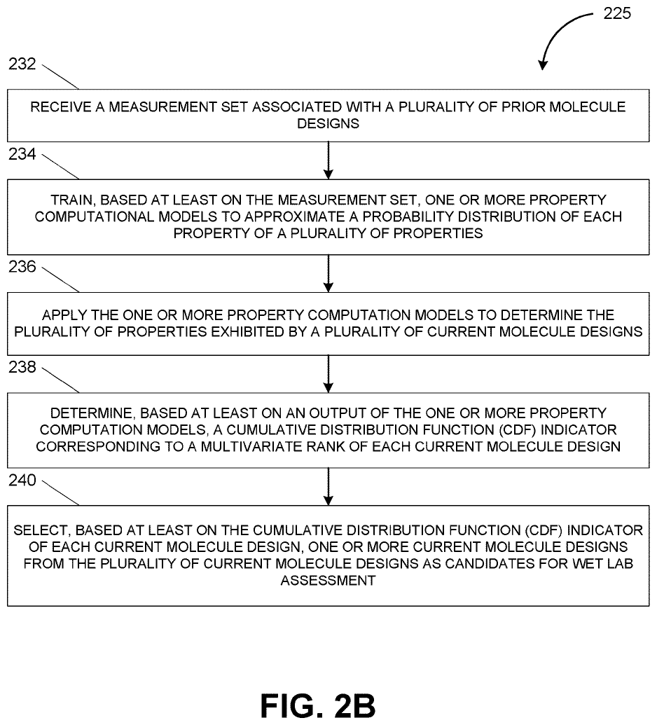

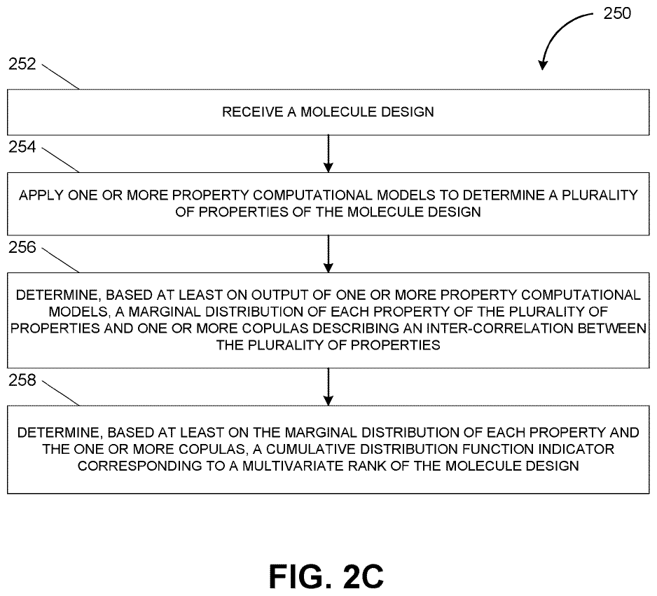

FIG. 2A depicts a flowchart illustrating an example of a process 200 for molecule design with multi-objective optimization, in accordance with some example embodiments. Referring to FIGS. 1-2A, the process 200 may be performed by the molecule design engine 110 and the selection engine 120 to identify, for example, a subset of the molecule designs generated by the molecule design engine 110 as candidates for wet lab assessment such as, for example, in vitro measurements, in vivo characterization, and/or the like.

At 202, a plurality of molecule designs are generated. In some example embodiments, a plurality of molecule designs including, for example, protein molecules or non-protein molecules such as small molecules, ions, nucleic acids, polysaccharides, glycolipids, and/or the like, may be generated. In some cases, each molecule design may be generated computationally, for example, by sampling a data distribution of molecules exhibiting one or more desirable properties (e.g., drug-like properties such as affinity, specificity, biological activity, developability, and/or the like). For example, in some cases, each molecule design may be generated by applying the molecule design computation model 115, which may be trained to approximate the data distribution including by at least determining a function (e.g., energy function, score function, and/or the like) whose output (e.g., energy value, score, and/or the like) differentiates between different density regions of the data distribution. As such, in some cases, each molecule design may be generated through multiple iterations of gradient-based Markov Chain Monte Carlo (MCMC) sampling (e.g., Langevin Markov Chain Monte Carlo (MCMC) sampling and/or the like). The sampling may be performed based on the output (e.g., score, energy value, and/or the like) of the function (e.g., score function, energy function, and/or the like) such that each successive iteration of sampling iteration includes drawing a sample (or molecule) from an incrementally higher density regions of the data distribution, which are more likely to be populated by molecules exhibiting the one or more desirable properties than lower density regions of the data distribution.

At 204, one or more property computation models may be applied to determine a plurality of properties exhibited by the plurality of molecule designs. In some example embodiments, the properties of each computationally generated molecule design may be determined by applying one or more property computation models trained to approximate the probability distribution of the possible values of each property. For example, in some cases, the one or more property computation models may be trained by at least updating a prior probability distribution of the possible values of each property (e.g., drug-like property) based on observations, such as wet lab measurements, of the properties exhibited by prior molecule designs. This training of the one or more property computation models may yield a posterior probability distribution of the possible values of each property. Accordingly, in some cases, the application of the one or more property computation models to determine the properties of a molecule design may include drawing multiple predictive samples from the posterior probability distribution of the possible values of each property. In some cases, the output of the one or more property computation models may include the predictive samples, each of which including a combination of values for the different properties of the molecule design. For instance, in instances where two objectives (or molecular properties of interest) are being optimized, each predictive sample output by the one or more property computation models may include a first value for a first property present in a molecule design and a second value for a second property present in the molecule design. It should be appreciated that the one or more property computation models may output multiple predictive samples for a single molecule design in order to reflect the uncertainty that is associated with inferring the properties of each molecule design. In other words, instead of the one or more property computation models outputting a single possible value for each property, the one or more property computation models may output multiple different values corresponding to the probability distribution of the possible values of each property. Doing so may be consistent with the observation that there may be some variability in the observed properties of each molecule design.

At 206, a cumulative distribution function (CDF) indicator corresponding to a multivariate rank is determined for each molecule design of the plurality of molecule designs based at least on an output of the one or more property computation models. In some example embodiments, for each molecule design, a utility metric corresponding to the probability of the molecule design being a Pareto-optimal solution (or a nondominated solution) may be determined. In the context of drug design, a molecule design may be a Pareto-optimal solution if none of the properties (e.g., drug-like properties) of the molecule design can be improved without degrading at least one other property of the molecule design. For example, in cases where the molecule design is being optimized for a first property as well as a second property, the molecule design may be a Pareto-optimal solution if the first value of the first property cannot be improved without degrading the second value of the second property.

In some example embodiments, an acquisition function may be applied to determine a utility metric for each molecule design. Conventional acquisition functions, such as improvement-based acquisition functions (e.g., expected hypervolume improvement (EHVI), noisy expected hypervolume improvement (NEHVI)) and entropy-based acquisition functions (e.g., max-value entropy search method (MESMO), joint entropy search (JES)), exhibit poor time complexity. Accordingly, in some cases, a multivariate ranking based acquisition function may be applied. In some cases, the multivariate ranking based acquisition function may be a cumulative distribution function (CDF) acquisition function in which the utility metric determined for each molecule design is a cumulative distribution function (CDF) indicator corresponding to the multivariate rank of the molecule design. As described in more details below, the cumulative distribution function (CDF) indicator of a molecule design may be consistent with the multivariate rank as well as the hypervolume (HV) bounded by the molecule design. That is, in instances where the molecule design is a Pareto-optimal solution, the cumulative distribution function (CDF) indicator of the molecule design and the corresponding hypervolume (HV) and multivariate rank may be more favorable than those of the molecule designs that it dominates. In some cases, the computational complexity of determining the cumulative distribution function (CDF) indicator may be further reduced by separately estimating the marginal distributions of the objectives being optimized (or molecular properties of interest) and one or more copulas (e.g., bivariate copulas) describing the inter-correlation therebetween.

At 208, one or more molecule designs from the plurality of molecule designs may be selected as candidates for wet lab assessment based at least on the cumulative distribution function (CDF) indicator of each molecule design. In some example embodiments, one or more of the molecule designs may be identified as candidates for synthesis and testing if the utility metrics (e.g., cumulative distribution function (CDF) indicator) of the molecule designs satisfy one or more thresholds. Alternatively and/or additionally, an N quantity of molecule designs having the best utility metric may be selected as candidates for synthesis and testing. In this latter case, one or more of the molecule designs may be selected as candidates for synthesis and testing if these molecule designs are part of the N quantity of molecule designs having the highest utility metric. In some cases, in addition to the utility metric associated with each molecule design, one or more additional conditions may be imposed. For instance, in the case of antibody design, the one or more molecule designs may be selected as candidates for synthesis and testing further based on the presence (or absence) of certain amino acid residues or sequences of amino acid residues (e.g., liability motifs).

FIG. 2B depicts a flowchart illustrating another example of a process 225 for molecule design with multi-objective optimization, in accordance with some example embodiments. Referring to FIGS. 1 and 2B, the process 225 may be performed, for example, by the selection engine 120 in order to select, over one or more successive iterations of multi-objective Bayesian optimization (MOBO), one or more molecule designs with incrementally better combination of properties for wet lab assessment (e.g., synthesis and testing by the one or more laboratory equipment 130).

At 232, a measurement set associated with a plurality of prior molecule designs is received. In some example embodiments, molecule designs may be optimized over multiple successive iterations of multi-objective Bayesian optimization (MOBO). In some cases, the properties (e.g., drug-like properties) of computationally generated molecule designs may be too expensive to evaluate, particularly when large quantities of molecule designs may be generated at once. Accordingly, in some cases, one or more property computation models (e.g., the property computation models 225) may be trained serve as in silico surrogates for determining the properties of the molecule designs. In some cases, the one or more property computation models may be trained to approximate, for each property, the probability distributions of the possible values of that property. For example, in some cases, the one or more property computation models may be trained using wet lab measurements of the properties of one or more molecule designs that were selected for synthesis and testing during a previous iteration of multi-objective Bayesian optimization (MOBO). As described in more details below, the properties of a molecule design may be determined by sampling combinations of values (e.g., one or more predictive samples) from these probability distributions. Moreover, these probability distributions may be further updated by retraining the property computation models with actual wet lab measurements obtained for the molecule designs.

At 234, one or more property computation models are trained, based at least on the measurement set, to approximate a probability distribution of each property of a plurality of properties. In some example embodiments, one or more property computation models may be trained to approximate the probability distribution of each property. For example, in some cases, a single property computation model may be trained to approximate the probability distribution of a single property or multiple properties. Alternatively and/or additionally, it is also possible for an ensemble of property computation models, which includes multiple independent property computation models, to be trained to approximate the probability distribution of a single property. The training of each property computation model may include updating, based at least on the measurements of the property exhibited by one or more molecule designs (e.g., from a previous iteration of multi-objective Bayesian optimization (MOBO)), a prior probability distribution the possible values of the property. Doing so may generate a posterior probability distribution of the possible value of the property. As described in more details below, once trained, the one or more property computation model may be applied to determine the properties of one or more molecule designs generated during a current iteration of multi-objective Bayesian optimization (MOBO).

At 236, the one or more property computation models are applied to determine the plurality of properties exhibited by a plurality of current molecule designs. In some example embodiments, the one or more property computation models may be applied to determine the properties of each molecule design generated during a current iteration of multi-objective Bayesian optimization (MOBO) by sampling from the probability distributions of the possible values of the properties. For example, in some cases, multiple predictive samples from the probability distributions may be generated for each molecule design from the current iteration of multi-objective Bayesian optimization (MOBO). Each predictive sample may include a value for each property including, for example, a first value of a first property and a second value of a second property present in a molecule design. As described in more details below, a cumulative distribution function (CDF) acquisition function may be applied to determine, based at least on the predictive samples generated by the one or more property computation models for each molecule design, a cumulative distribution function (CDF) indicator corresponding to an expected multivariate rank of the molecule design.

At 238, a cumulative distribution function (CDF) indicator corresponding to a multivariate rank may be determined, based at least on an output of the one or more property computation models, for each current molecule design. In some example embodiments, a cumulative distribution function (CDF) acquisition function may be applied in order to determine, for each molecule design generated during a current iteration of multi-objective Bayesian optimization (MOBO), a cumulative distribution function (CDF) indicator corresponding to an expected multivariate rank of the molecule design. In some cases, the expected multivariate rank of a molecule design may rank, based on a combination of properties (e.g., drug-like properties), the molecule design against the other molecule designs that are generated during the current iteration of multi-objective Bayesian optimization. Furthermore, the expected multivariate rank of the molecule design may account for the uncertainty that may be present in the output of the property computation models which, as noted, may be probabilistic surrogate models. In some cases, the expected multivariate rank of the molecule design may correspond to the probability of the molecule design being one of the Pareto-optimal solutions populating the Pareto frontier. That is, a first molecule design with a multivariate rank that is superior to that of a second molecule design may be more proximate to the Pareto frontier or is more likely to be a Pareto-optimal solution than the second molecule design. Accordingly, in some cases, which molecule designs generated during the current iteration of multi-objective Bayesian optimization (MOBO) are selected for wet lab assessment (e.g., synthesis and testing) may be contingent on the respective multivariate rankings of each molecule design.

As described in more details below, in some example embodiments, the cumulative distribution function (CDF) indicator of a molecule design may be estimated using one or more copulas. For example, in some cases, a marginal distribution of each property and one or more copulas described an inter-correlation between the properties may be determined based on the predictive samples output by the property computation model for each molecule design. Furthermore, in some cases, the cumulative distribution function (CDR) indicator of each molecule design may be determined based on the marginal distribution of each property and the corresponding copulas. Alternatively, other cumulative distribution function (CDF) estimators may be used instead of the aforementioned copulas. A multivariate Gaussian cumulative distribution function (CDF) is one example in which a mean (p) and covariance (E) of the training data (e.g., the measurement set received in operation 232) are determined before a closed-form analytical solution is used to obtain a multivariate Gaussian distribution for computing the cumulative distribution function (CDF) indicator \hat{F}(x)=P(X≤x) where X˜(μ, Σ). In the case of empirical cumulative distribution function (CDF), the estimator may be a step function that jumps by

$$\frac{1}{n}$$

at each of the n data points. As shown in Equation (1) below, the value of the empirical cumulative distribution function (CDF) may be a fraction of observations of the measured variable (e.g., in the measurement set received in operation 232) that are less than or equal to the specified value.

$${\hat{F}}_{n} ( x ) = \frac{\#elements in sample < t}{n} = \frac{1}{n} {\sum }_{i = 1}^{n} {1}_{{X}_{i} < t} \qquad ( 1 )$$

Another example of a cumulative distribution function (CDF) estimator that can be used is kernel density estimation (KDE), which is a mixture of density estimator. Since the density is

$$\hat{f} ( x ) = \frac{1}{m} {\sum }_{i = 1}^{M} {f}_{i} ( x ) ,$$

then the joint cumulative distribution function (CDF) may be a mixture of cumulative density functions,

$$\hat{F} ( x ) = \frac{1}{m} {\sum }_{i = 1}^{M} {F}_{i} ( x ) .$$

A Gaussian kernel may be expressed as

$${f}_{i} ( x ) = \frac{ϕ ( x - {x}^{′} )}{σ}$$

and analogously

$${F}_{i} ( x ) = \frac{Φ ( x - {x}^{′} )}{σ}$$

where σ denotes the kernel bandwidth.

At 240, one or more current molecule designs from the plurality of current molecule designs may be selected, based at least on the cumulative distribution function (CDF) indicator of each current molecule design, as candidates for wet lab assessment. In some example embodiments, the cumulative distribution function (CDF) acquisition function may balance the exploration of uncertain molecule designs and exploitation of those likely to maximize the objectives when selecting, for wet lab assessment, one or more of the molecule designs generated during the current iteration of multi-objective Bayesian optimization (MOBO). In some cases, one or more of the molecule designs generated during the current iteration of multi-objective Bayesian optimization (MOBO) may be selected for wet lab assessment based at least on the cumulative distribution function (CDF) indicator of each molecule design. For example, in some cases, the cumulative distribution function (CDF) indicator of a molecule design may be a value between [0,1]. The molecule designs that are generated during the current iteration of multi-objective Bayesian optimization may be ranked based on their respective cumulative distribution function (CDF) indicator and a threshold quantity (e.g., an N quantity) of those with the best cumulative distribution function (CDF) indicator (or best expected multivariate ranking) may be selected for wet lab assessment. Alternatively and/or additionally, the molecule designs that are selected for wet lab assessment may be required to exhibit a cumulative distribution function (CDF) indicator (or expected multivariate ranking) that satisfies one or more thresholds. As noted, the wet lab measurements of those molecule designs selected for wet lab assessment during this current iteration of multi-objective Bayesian optimization (MOBO) may be used to further update the one or more property computation models during a subsequent iteration of multi-objective Bayesian optimization (MOBO).

FIG. 2C depicts a flowchart illustrating another example of a process 250 for molecule design with multi-objective optimization, in accordance with some example embodiments. Referring to FIGS. 1 and 2A-2C, the process 250 may be performed, for example, by the selection engine 120 to determine the utility metric of each molecule design generated by the molecule design engine 110. In some cases, the process 250 may implement at least a portion of operation 206 of the process 200 shown in FIG. 2A or operation 238 of the process 225 shown in FIG. 2B.

At 252, a molecule design is received. In some example embodiments, one or more molecule designs generated computationally by sampling a data distribution of molecules exhibiting one or more desirable properties (e.g., drug-like properties) may be received. In some cases, each of the molecule designs may correspond to a protein molecule or, alternately, a non-protein molecule such as a small molecule, an ion, a nucleic acid, a polysaccharide, a glycolipid, and/or the like.

At 254, one or more property computation models are applied to determine a plurality of properties of the molecule design. In some example embodiments, the molecule designs may be optimized for multiple objectives (or molecular properties of interest) such as, for example, a first property and a second property. In some cases, certain practical limitations, such as the limited availability and exorbitant cost of laboratory resources, may prevent every computationally generated molecule design from undergoing wet lab assessment. Accordingly, in some cases, one or more iterations of multi-objective Bayesian optimization (MOBO) may be performed. In some cases, multi-objective Bayesian optimization (MOBO) may be a sequential design strategy in which molecule designs with incrementally better combinations of the first property and the second property are selected for wet lab assessment. For example, in some cases, a current molecule design that is selected for wet lab assessment during a current iteration of multi-objective Bayesian optimization (MOBO) may exhibit a better combination of the first property and the second property than one or more baseline molecule designs, which may include at least one previous molecule design that was selected for wet lab assessment during a previous iteration of multi-objective Bayesian optimization (MOBO).

In some cases, the objective functions for the first property and the second property may be unknown black-box functions that are too expensive to evaluate. As such, the in some cases, one or more property computation models may be trained to serve as in silico surrogates for the objective functions. That is, one or more property computation models may be one or more machine learning models trained to determine the first property and the second property of each molecule design generated computationally during each iteration of multi-objective Bayesian optimization (MOBO). For example, in some cases, a first property computation model trained to approximate a first probability distribution of the possible values of the first property and a second property computation model trained to approximate a second probability distribution of the possible values of the second property may be applied. In some cases, the first property computation model may be applied to determine, based at least on the first probability distribution, one or more values of the first property present in a molecule design that is generated computationally during a current iteration of multi-objective Bayesian optimization (MOBO). Meanwhile, the second property computation model may be applied to determine, based at least on the second probability distribution, one or more values of the second property present in the same molecule design. The outputs of the first property computation model and the second property computation model may form one or more predictive samples, each of which including a first value of the first property and a second value of the second property exhibited by the molecule design having the first value of the first property.

It should be appreciated that in some cases, a single property computation model may be trained and applied to determine multiple properties of each molecule design. Alternately, in some cases, it is also possible that more than one property computation model, such as an ensemble of property computation models, may be applied to determine the value of a single property of each molecule design. The inclusion of multiple property computation models (or an ensemble of property computation models) for a single property may compensate for at least some of the uncertainty that may be present in the output of individual property computation models. For example, in some cases, the output of a first property computation model may be more uncertain (or have a lower confidence of being accurate) for a particular molecule design while the output of a second property computation model may be less uncertain (or have a higher confidence of being accurate) for the same molecule design. As such, when multiple property computation models (or an ensemble of property computation models) are applied to determine the property of a molecule design, the lower uncertainty in the output of some property computation models may compensate for the higher uncertainty in the output of other property computation models.

In some example embodiments, the noise that may be present in observed properties of one or more baseline molecule designs may be compensated for in a variety of ways. For example, in cases where the cumulative distribution function (CDF) indicator corresponds to an expected multivariate ranking of a molecule design relative to the multivariate ranking of one or more baseline molecule designs, the values of the properties present in the one or more baseline molecule designs may be observed in a wet lab. However, these observed values may include at least some noise arising from measurement errors. As such, in some cases, the effects of this noise may be reduced (or minimized) by determining the utility metric of each baseline molecule design based on the outputs of the one or more property computations models after the one or more property computation models have been retrained based on the measured values. For instance, the retrained property computation models may be applied to determine the values of the properties present in the molecule design as well as the values of the properties present in the one or more baseline molecule designs. The utility metric for the molecule design and that of each baseline molecule design may be determined based on the outputs of the retrained property computation models instead of the observed values.

At 256, a marginal distribution of each property of the plurality of properties and one or more copulas describing an inter-correlation between the plurality of properties based at least on an output of the one or more property computation models. In some example embodiments, a cumulative distribution function (CDF) acquisition function may be applied to determine, for each computationally generated molecule design, a utility metric indicating the probability of the molecule design being a Pareto-optimal solution (or a non-dominated solution). As noted, in some cases, a molecule design that qualifies as a Pareto-optimal solution may be a molecule design whose individual properties (e.g., drug-like properties) cannot be further improved without degrading at least one such property. In some cases, the cumulative distribution function (CDF) acquisition function may be applied instead of a conventional acquisition function (e.g., improvement-based acquisition function, entropy-based acquisition function, and/or the like). The application of the cumulative distribution function (CDF) acquisition function may generate a cumulative distribution function (CDF) indicator for each computationally generated molecule design. As noted, in some cases, the cumulative distribution function (CDF) indicator for a molecule design may correspond to its multivariate rank, or a ranking of the molecule design based on multiple objectives (or molecular properties of interest) relative to one or more other molecule designs (e.g., from the same design iteration) and/or baseline molecule designs (e.g., from previous design iterations). Moreover, the cumulative distribution function (CDF) indicator for the molecule design may also correspond to the hypervolume (HV) bounded by the properties of the molecule design, which is a utility metric associated with a conventional improvement-based acquisition function.

In some example embodiments, copulas, such as bivariate copulas, may be used to estimate the cumulative distribution function (CDF) indicator of a molecule design. For example, in some cases, the cumulative distribution function (CDF) indicator of a computationally generated molecule design may be determined by at least decomposing the multivariate joint distribution of multiple objectives (or molecular properties of interest) into the marginal distributions of each individual objective (or molecular property of interest) and one or more copulas (e.g., bivariate copulas). The marginal distribution of an objective (or molecular property of interest) in a multivariate joint distribution may specify the distribution of values for that objective and is agnostic to the values of the other objectives in the multivariate joint distribution. Meanwhile, a copula is a coupling function describing a dependence structure (e.g., a copula matrix) specifying the inter-correlation between the marginal distributions of two or more objectives (or molecular properties of interest). It should be appreciated that each of the aforementioned marginal distributions and copulas may be determined separately from one another. As such, using copulas to estimate the cumulative distribution function (CDF) indicator of the molecule design may obviate the computational complexity of determining the underlying multivariate joint distribution, which would require estimating the multivariate joint density function and computing the integral thereof.

In some example embodiments, the computational complexity of estimating the cumulative distribution function (CDF) indicator of each molecule design may be further reduced by truncating certain copulas, such as those representative of higher order dependencies between some objectives (or molecular properties of interest) when those dependencies are sufficiently trivial to be ignored when ranking two or more molecule designs. In some cases, the truncation of some copulas may avoid the removal of some dependencies, such as those corresponding to certain experimental dependencies, biological dependencies, or preferences for particular types of molecule designs. For example, the copula coupling a first marginal distribution of expression level and a second marginal distribution of binding affinity may be preserved during truncation at least because the experimental dependency in which the expression level of a molecule design (e.g., in cell culture) may be required to satisfy some threshold (e.g., in mass per volume) in order for the molecule design to be produced in sufficient quantities for subsequent assays its binding affinity to a target antigen. In other words, in some cases, the truncation of copulas, particularly the selection of copulas that are preserved, may enable the enforcement of dependencies between certain objectives (or molecular properties of interest). Nevertheless, the removal of at least some copulas as part of the truncation process may further increase the computational efficiency of using copulas as an estimator for the cumulative distribution function (CDF) indicator of computationally generated molecule designs.

In some example embodiments, the multivariate joint distribution of two or more objectives (or molecular properties of interest) may be decomposed into one or more bivariate joint distributions, each of which including a paired grouping of the objectives. For example, for an M quantity of objectives (or molecular properties of interest) and in cases where M>2, the corresponding M-dimensional joint distribution may be factorized into a collection of bivariate copulas. In some cases, pairwise factorization of the M-dimensional joint distribution may be performed to generate a structure called a vine, which in this case may be a hierarchical model including a sequence of M−1 nested trees linked by an

$$\frac{M ( M - 1 )}{2}$$

number of bivariate copulas. The m-th tree Tₘ may include a set of nodes Vₘ interconnected by a set of edges Eₘ (e.g., Tₘ=(Vₘ, Eₘ). A node in the tree Tₘ may be representative of at least one objective being optimized (or molecular property of interest) or a combination thereof. Meanwhile, an edge e interconnecting two nodes in the tree may be representative of a bivariate copula c coupling the marginal distributions of the objectives associated with each node. Accordingly, a vine may include a set of trees Tₘ=(Vₘ, Eₘ) for m∈[M−1] and a set of pair-copulas tree cⱼ_{ₑ}_{,k}_{ₑ}_{|D}_{ₑ} for e∈Eₘ and m∈[M−1]. It should be appreciated that the factorization may not be unique, meaning that the same M-dimensional joint distribution may be decomposed into different collections of bivariate copulas.

In some example embodiments, the hierarchical nature of the vine may enable the enforcement of dependencies between different objectives (or molecular properties of interest). For example, in some cases, the hierarchical structure of the vine may encode a partial ordering in which some objectives (or molecular properties of interest) are prioritized over others. This partial ordering may be consistent with certain experimental dependencies in which one property of a molecule design must satisfy one or more criteria before its other properties can be measured. For instance, in the case of antibody design, if the expression level of a molecule design (e.g., in cell culture) fails to satisfy some threshold (e.g., in mass per volume), the molecule design cannot be produced in sufficient quantities for subsequent assays for other properties, such as binding affinity to a target antigen. To capture this experimental dependency, the hierarchical structure of the corresponding vine may encode a partial ordering in which expression is prioritized over affinity (e.g., expression→affinity). This may mean that a bivariate copula coupling the marginal distributions of expression and affinity may exclude expression levels that fail to satisfy certain thresholds. In practice, it should be noted that experimental dependencies can create an asymmetry amongst the objectives (or molecular properties of interest). In the foregoing example, less data may be available for molecule designs with insufficient expression levels because no affinity measurements are available for these designs.

In some cases, a partial ordering in which some objectives (or molecular properties of interest) are prioritized over others may reflect a preference for certain types of molecule designs. For example, in some cases, one or more properties may be prioritized to prevent molecule designs that perform poorly with respect to these properties from advancing no matter how well the molecule designs perform in other properties. In the context of antibody body design, if a molecule design does not bind to the target antigen, it has failed in its primary function and there would be little interest to explore its developability properties, such as specificity to the target antigen and thermostability. This may be true even though, unlike for molecule designs with inadequate expression levels, these developability properties are often still measurable. Moreover, in some cases, a partial ordering capturing this preference in addition to the foregoing experimental dependency may be encoded in the hierarchical structure of the corresponding vine with expression being prioritized over affinity and affinity being further prioritized over developability properties such as specificity and thermostability (e.g., expression→affinity→{specificity, thermostability}).

At 258, a cumulative distribution function (CDF) indicator corresponding to a multivariate rank of the molecule design is determined based at least on the marginal distribution of each property and the one or more copulas. In some example embodiments, copulas (e.g., bivariate copulas) may be used to estimate the cumulative distribution function (CDF) indicator of each computationally generated molecule design. For example, in some cases, the cumulative distribution function (CDF) indicator of a molecule design may be determined based at least on the marginal distribution of each objective (or molecular property of interest) and the copulas (e.g., bivariate copulas) describing the inter-correlation between the objectives. As noted, doing so may reduce the computational complexity of estimating the underlying multivariate joint distribution at least because the marginal distributions and the corresponding copulas may be determined separately. Moreover, the cumulative distribution function (CDF) of the molecule design may be associated with a more interpretable scale than conventional utility metrics such as hypervolume (HV) indicators, whose scales do not carry any information about the internal ordering of different molecule designs. For instance, in some cases, the cumulative distribution function (CDF) indicator of a molecule design may be a value between 0 and 1. In some cases, the closer the value of the cumulative distribution function (CDF) indicator is to 1, the more likely the molecule design is to be a Pareto-optimal solution that is proximate to the Pareto frontier. In other words, in some cases, the cumulative distribution function (CDF) indicator of the molecule design may be sufficient to determine its candidacy for wet lab assessment, without necessarily requiring any comparison to the cumulative distribution function (CDF) indicators of other molecule designs.

As notes, in some cases, the cumulative distribution function (CDF) indicator of each molecule design may correspond to its multivariate rank, which is indicative of the distance to a Pareto front populated by Pareto-optimal solutions in the form of molecule designs whose properties cannot be further improved without degrading at least one such property. Accordingly, in some cases, by determining the cumulative distribution function (CDF) indicator of each molecule design generated by the molecule design engine 110, the selection engine 120 may identify those that are more likely to be Pareto-optimal solutions. In some cases, molecule designs that are Pareto-optimal solutions on the Pareto frontier may be associated with the same (or tied) multivariate ranks, meaning that corresponding cumulative distribution function (CDF) indicators are also the same (or tied).

In some example embodiments, one or more molecule designs that meet specific criteria with respect to one or more properties (e.g., drug-like properties) may be identified for wet lab assessment (e.g., synthesis, testing, and/or the like). In some cases, the properties of molecule design may be optimized, through active learning, over multiple successive design iterations. For example, in some cases, the one or more molecule designs that are selected during a current iteration of multi-objective Bayesian optimization (MOBO) may exhibit a better combination of properties (e.g., drug-like properties) than one or more baseline molecule designs. In some cases, the one or more baseline molecule designs may be one or more prior molecule designs, which were selected for wet lab assessment during a previous iteration of multi-objective Bayesian optimization. As such, in some cases, the properties of the one or more baseline molecule designs may have been observed in the wet lab, for example, through in vitro measurements, in vivo characterization, and/or the like. In some cases, the cumulative distribution function (CDF) indicator of a molecule design from a current iteration of multi-objective Bayesian optimization (MOBO) may correspond to an expectation that the molecule design exhibits a better combination of properties than those baseline molecule designs from the previous iteration of multi-objective Bayesian optimization (MOBO).

As noted, the observed values of the properties of the one or more baseline molecule designs may be contaminated by noise arising, for example, from measurement errors present in the laboratory equipment. Accordingly, in some cases, the effects of this noise may be reduced (or minimized) by at least retraining the one or more property computation models during a subsequent iteration of multi-objective Bayesian optimization (MOBO) in order to refit the one or more property computation models with additional observations obtained for molecule designs generated during one or more previous iterations of multi-objective Bayesian optimization (MOBO). For example, in some cases, the one or more property computation models may be retrained based on the observed values of the properties of the one or more baseline molecule designs. In some cases, the one or more property computation models may be probabilistic surrogate models that accounts for the uncertainty in wet lab measurements by at least approximating a probability distribution (e.g., a probability density function (PDF)) of the possible values of each property (e.g., drug-like property). For instance, in some cases, the retraining of a property computation model may include updating a prior probability distribution of the possible values of a property such that the retrained property computation model determines the property of a molecule design based on the resulting posterior probability distribution. In some cases, the retrained property computation models may be applied to determine the properties of the baseline molecule designs as well the molecule designs generated during the current iteration of multi-objective Bayesian optimization (MOBO).

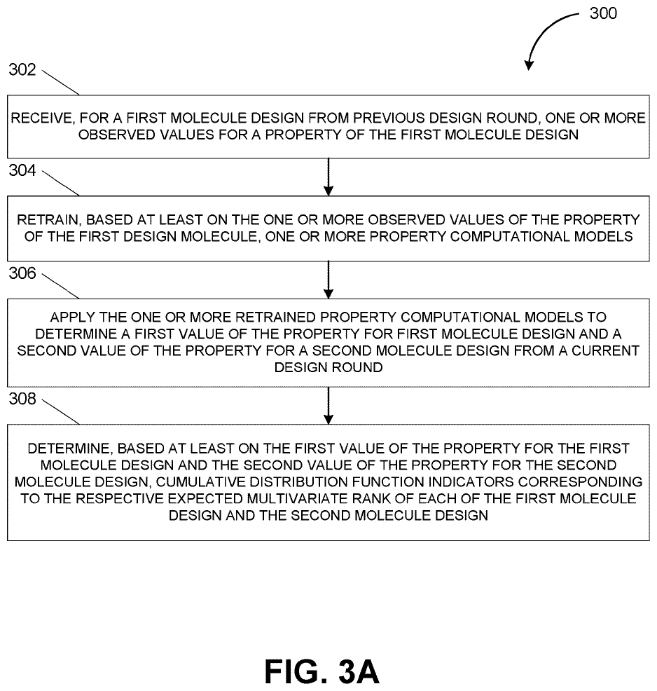

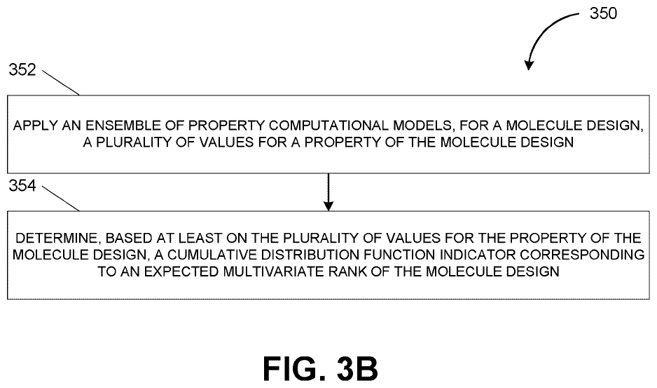

To further illustrate, FIG. 3A depicts a flowchart illustrating an example of a process 300 for determining one or more properties of a molecule design, in accordance with some example embodiments. Referring to FIGS. 1, 2A-2B, and 3A, the process 300 may be performed by the selection engine 120 and may implement, for example, at least a portion of operation 206 of the process 200 shown in FIG. 2A or at least a portion of operation 238 of the process 225 shown in FIG. 2B.

At 302, one or more observed values for a property of a first molecule design from a previous design iteration may be received. In some example embodiments, one or more of the molecule designs generated during a current iteration of multi-objective Bayesian optimization (MOBO) may be selected for wet lab assessment (e.g., in vitro measurements, in vivo characterization, and/or the like). In some cases, molecule designs that are selected may exhibit a better combination of properties (e.g., drug-like properties) than one or more baseline molecules. For example, in some cases, a molecule design may be selected based at least on a comparison between its utility metric and that of each baseline molecule design. As noted, in some cases, the aforementioned utility metric may be a cumulative distribution function (CDF) indicator corresponding to the expected multivariate rank of each molecule design. For instance, in some cases, the molecule designs that are Pareto-optimal solutions may have a tied multivariate rank that is better than the multivariate ranks of the molecule designs with an inferior combination of properties (e.g., drug-like properties).

In some cases, at least some of the baseline molecule designs may be molecule designs that have been selected for wet lab assessment during one or more previous design iterations. As such, the values of the properties (e.g., drug-like properties) of the one or more baseline molecule designs may have been observed in a wet lab. For example, the observed values that are available for the properties of each baseline molecule design may include one or more in vitro measurements and/or in vivo characterization. However, as described in more details below, the observed values of the properties of these baseline molecule designs, which may be used during the current iteration of multi-objective Bayesian optimization (MOBO) to identify one or more molecule designs with a better combination of properties, may be contaminated with noise (e.g., arising from measurement errors associated with laboratory equipment and/or the like).

At 304, one or more property computation models are retrained based at least on the one or more observed value of the property of the first molecule design. In some example embodiments, the one or more property computation models may be probabilistic surrogate models that accounts for the uncertainty in wet lab measurements by at least approximating a probability distribution (or probability density function (PDF)) of the possible values of each property (e.g., drug-like property). As noted, in some cases, the observed values of the properties of the one or more baseline molecule designs may be contaminated with noise arising, for example, from measurement errors present in laboratory equipment and/or the like. This noise may distort the utility metrics computed directly based on the observed values of the properties of each baseline molecule design. Accordingly, in some cases, the observed values of the properties of the baseline molecule designs directly may not be used directly. Instead, in some cases, the effect of the noise that may be present in the observed values of the properties of the baseline molecule designs received in operation 302 may be reduced (or minimized) by at least retraining the one or more property computation models based on these observed values. Doing so may update the prior probability distribution of the possible values of these properties based on the observed values such that the retrained property computation models determines the properties of one or more molecule designs based on the resulting posterior probability distribution. For example, as noted, the property computation models may output, for a molecule design, one or more predictive samples. In some cases, a single predictive sample may include a first value of a first property determined based on the posterior probability distribution of the possible values of the first property and a second value of a second property determined based on the posterior probability distribution of the possible values of the second property.

At 306, the one or more retrained property computation models are applied to determine a first value of the property for the first molecule design and a second value of the property for a second molecule design from a current design iteration. In some example embodiments, the retrained property computation models may be applied to determine the values of the properties for the baseline molecule designs (e.g., from one or more previous iterations of multi-objective Bayesian optimization (MOBO)) as well as the values of the properties for the molecule designs generated during the current iteration of multi-objective Bayesian optimization (MOBO). The retrained property computation models may be associated with posterior probability distributions that have been updated to reflect the observed values of the properties of the baseline molecule designs including, in some cases, any of the noise that may be present therein. In the case of expression level, for example, the retrained property computation models may be applied to determine a first expression level of a first molecule design from a previous design iteration as well as a second expression level of a second molecule design from the current design iteration. Although the expression level of the first molecule design has been observed through wet lab experiments, the utility metric (e.g., the cumulative distribution function (CDF) indicator) of the first molecule design is not determined directly based on the observed expression level of the baseline molecule design. Instead, as described in more details below, the utility metric of the first molecule design from the previous design iteration as well as the utility metric of the second molecule design from the current design iteration may be determined based on the respective expression levels of each molecule design determined by the retrained property computation model.

At 308, cumulative distribution function (CDF) indicators corresponding to the respective expected multivariate ranks of each of the first molecule design and the second molecule design are determined based at least on the first value of the property for the first molecule design and the second value of the property for the second molecule design. In some example embodiments, a first cumulative distribution function (CDF) indicator corresponding to a first expected multivariate rank of the first molecule design from the previous iteration of multi-objective Bayesian optimization (MOBO) may be determined. Furthermore, in some cases, a second cumulative distribution function (CDF) indicator corresponding to a second expected multivariate rank of the second molecule design from the current iteration of multi-objective Bayesian optimization (MOBO) may also be determined. In some cases, the first cumulative distribution function (CDF) indicator of the first molecule design may be determined based on the values of the properties of the first molecule design determined by the retrained property computation models and not the observed values for these properties. As noted, this may be done to reduce (or minimize) the effect that the noise present in the observed values may have on the utility metric computed directly therefrom. Returning to the expression level example, the first cumulative distribution function (CDF) indicator of the first molecule design from the previous iteration of multi-objective Bayesian optimization may be determined based on the first expression level determined by the retrained property computation models while the second cumulative distribution function (CDF) indicator of the second molecule design from the current iteration of multi-objective Bayesian optimization (MOBO) may be determined based on the second expression level determined by the retrained property computation models. In some cases, the first cumulative distribution function (CDF) indicator and the second cumulative distribution function (CDF) indicator may capture the distance (or proximity) between the corresponding molecule designs and the true Pareto frontier. A molecule design with a better cumulative distribution function (CDF) indicator, which in this case may correspond to a better expected multivariate rank, may be closer to the true Pareto frontier and is therefore more likely to be a Pareto-optimal solution. In some cases, the second molecule design may be selected for wet lab assessment if the second cumulative distribution function (CDF) indicator of the second molecule design is better than that of the first molecule design and, in some cases, a third cumulative distribution function (CDF) indicator of a third molecule design generated during the current iteration of multi-objective Bayesian optimization (MOBO).

In some example embodiments, instead of or in addition to retraining the property design computational models, the uncertainty in the output of each property computation model may also be reduced (or minimized) by at least leveraging multiple property computation models (e.g., an ensemble of property computation models) for each property. For example, as described in more details below, the selection engine 120 may apply a first property computation model and a second property computation model to assess a single property of a molecule design such as, for each, individual drug-like properties such as affinity, specificity, biological activity, developability, and/or the like. As such, the value of that property, which is used towards computing the utility metric of the molecule design, may be determined based on a first value of the property determined by the first property computation model and a second value of the same property determined by the second property computation model.

To further illustrate, FIG. 3B depicts a flowchart illustrating another example of a process 350 for determining one or more properties of a molecule design, in accordance with some example embodiments. Referring to FIGS. 1, 2A-C, and 3A-B, the process 350 may be performed by the selection engine 120 and may implement, for example, at least a portion of operation 204 of the process 200 shown in FIG. 2A, operation 236 of the process 225 shown in FIG. 2B, operation 254 of the process 250 shown in FIG. 2C or, in some cases, operation 306 of the process 300 shown in FIG. 3A.

At 352, a first property computation model is applied to determine a first value of a property exhibited by a molecule design and a second property computation model to determine a second value of the property exhibited by the molecule design. In some example embodiments, to determine the value of an individual property present in a molecule design, multiple property computation models (or an ensemble of property computation models) may be applied. For example, in some cases, an ensemble of property computation models that includes a first property computation model and a second property computational may be applied. In this example, each property computation model in the ensemble may be trained to determine the same property (e.g., drug-like property). Accordingly, the first property computation model may output a first value for the property present in the molecule design while the second property computation model may output a second value for the same property present in the molecule design. In some cases, the first value and the second value of the property may each be determined based on a probability distribution, which enumerates the probability of occurrence of each possible value of the corresponding property. For instance, the output of the first property computation model may include a first value determined based on a first probability distribution across the range of possible values for the property (e.g., drug-like property) while the output of the second property computation model may include a second value determined based on a second probability distribution across the range of possible values for the same property. In some cases, the output of each of the first property computation model and the second property computation model may be zero-inflated, meaning that the output includes one value (e.g., a binary value) indicating whether the value of the property satisfies a certain threshold and another value indicating the actual value of the property present in the molecule design.

At 354, a third value of the property exhibited by the molecule design for computing a cumulative distribution function (CDF) indicator corresponding to an expected multivariate rank of the molecule design is determined based at least on the first value and the second value. In some example embodiments, the outputs of multiple property computation models may be leveraged in order to reduce (or minimize) the uncertainty that may be present in the output of each individual property computation model. For example, in some cases, differences between the first property computation model and the second property computation model, such as architecture and training, may cause one property computation model to be more (or less) certain than the other when applied to the same molecule design. In some cases, for example, the first property computation model may generate a more certain output for a first molecule design than the second property computation model but the second property computation model may generate a more certain output for a second molecule design than the first property computation model. Accordingly, in some cases, the outputs of multiple property computation models may be leveraged when determining the utility metric of a molecule design in order to compensate for this uncertainty. That is, in some cases, multiple values of the same property as determined by the ensemble of property computation models may be used to determine the utility metric (e.g., cumulative distribution function (CDF) indicator) of each molecule design generated by the molecule design engine 110. For instance, in some cases, the utility metric of each molecule design may be determined based on a mean, a median, a maximum, a minimum, a mode, and/or a range of the outputs generated by the ensemble of property computation models. As such, in cases where the first property computation model is applied to determine a first value of the property present in a molecule design and the second property computation model is applied to determine a second value of the same property, a third value for the property may be determined based at least on the first value and the second value. In some cases, the third value of the property may correspond to a mean, a median, a maximum, a minimum, a mode, and/or a range of the first value and the second value. Moreover, in some cases, the cumulative distribution function (CDF) indicator of the molecule design, which corresponds to its expected multivariate rank, may be determined based on the third value instead of any individual one of the first value and the second value.

In some example embodiments, multi-objective Bayesian optimization (BO) may be performed to trade off exploration (evaluating highly uncertain molecule designs) and exploitation (evaluating molecule designs believed to increase or maximize the objectives) by leveraging one or more property computation models and a multivariate rank based acquisition function. In the context of drug design, each objective (or molecular property of interest) f: χ→ [symbol] may be considered a black-box function that is too expensive to evaluate (e.g., in the wet lab) for the molecule designs sampled from the design space χ. As such, the goal of Bayesian optimization (BO) is to efficiently identify a molecule design x*∈F that increases (or maximizes) each individual objective (or molecular property of interest) f.

In some cases, Bayesian optimization (BO) may include leveraging the one or more property computation models, which serve as probabilistic surrogate models by performing in silico evaluations of each objective f. Moreover, in some cases, Bayesian optimization (BO) may include applying a multivariate rank based acquisition function, such as a cumulative distribution function (CDF) acquisition function, to evaluate, based on the outputs of the one or more property computation models, each molecule design x. As noted, in some cases, the cumulative distribution function (CDF) acquisition function may be applied to determine, for each molecule design x, a cumulative distribution function (CDF) indicator corresponding to an expected multivariate rank of the molecule design x. For example, in some cases, the cumulative distribution function (CDF) indicator of a molecule design x may quantify the quality of the molecule design x as a Pareto-optimal solution that has been optimized across multiple objectives f. In some cases, the cumulative distribution function (CDF) indicator of the molecule designs x may inform the tradeoff between exploring the design space χ to evaluate more uncertain molecule designs (e.g., molecule designs with unknown likelihood of increasing or maximizing the objective f) and exploiting of more certain molecule designs (e.g., molecule designs with greater likelihood of increasing or maximizing the objective f).

In some cases, the property computation model \hat{f}: [symbol] → [symbol] may approximate, based on existing information such as observed values of the objective f, a prior probability distribution of the values of the objective f. For example, where f is the expression level of a molecule design x, the property computation model \hat{f}:χ→ [symbol] may be trained based on web lab measurements of the expression level exhibited by known molecule designs including, in some cases, molecule designs from previous design iterations. Given the presence of observation noise (e.g., measurement errors associated with laboratory equipment and/or the like), the property computation model \hat{f}: [symbol] → [symbol] may be trained on a noisy dataset available up to a given design iteration t. In other words, each iteration t E N may be associated with a dataset Dₜ={(x⁽¹⁾, y⁽¹⁾), (x^{(N}^{ₜ}⁾, y^{(N}^{ₜ}⁾)}∈Dₜ where each y⁽ⁿ⁾ is a noisy observation of the objective f. The property computation model \hat{f}: [symbol] → [symbol] may be retrained (or refit), for example, at the current design iteration Dₜ, to further update the prior probability distribution of the values of the objective f based on the dataset from the previous design iteration Dₜ₋₁. Accordingly, the property computation model \hat{f}: [symbol] → [symbol] may be trained (and retrained) to infer the posterior probability distribution p(\hat{f}|Dₜ), which quantifies the plausibility of surrogate objectives \hat{f}∈ [symbol] . In the expression level example, the posterior distribution p(\hat{f}|Dₜ) quantifies the probability distribution of possible expression levels exhibited by the next batch of molecule designs.

An example of the acquisition function α(x) is shown as Equation (2) below. With a conventional acquisition function, such as an improvement-based acquisition function, the integral in Equation (2) may be approximated by Monte Carlo (MC) with posterior samples \hat{f}^{(j)}˜p(f| [symbol] ₜ) but is computationally taxing. In some cases, a molecule design that maximizes the acquisition function α(x) be selected for wet lab assessment of the objective f before the property computation models are refit on the dataset Dₜ augmented with the observations.

$$a ( x ) = \int u ( x , \hat{f} , {𝒟}_{t} ) p ( \hat{f} \vert {𝒟}_{t} ) d \hat{f} \qquad ( 2 )$$

When there is a single objective (or molecular property of interest), the best molecule design may be identified based on a ranking of the property values. When there are multiple objectives (or molecular properties of interest), the best molecule design may not be one having the best values for every objective (or property) at least because a single molecule design that excels in every objective f may not exist. Suppose there are M objectives, f: [symbol] → [symbol] ^{M}. The goal of multi-objective Bayesian optimization (BO) in this paradigm may be identify the set of Pareto-optimal solutions such that improving one of the M objectives leads to the worsening of another. A molecule design x may dominate another molecule design x′, or f(x) [symbol] f(x′), if fₘ(x)≥fₘ(x′) for all m∈[1, . . . , M} and fₘ(x)>f(x′) for some m. The set of non-dominated solutions [symbol] * may be defined in terms of the Pareto frontier (PF) [symbol] * as indicated in Equation (3) below. It should be appreciated that although the set of non-dominated solutions [symbol] * may be infinite, multi-objective Bayesian optimization (BO) may seek to identify a finite subset of thereof within, for example, a threshold quantity of design iterations.

$${𝒳}^{⋆} = \{ x : f ( x ) ∈ {𝒫}^{*} \} , \\ where {𝒫}^{*} = \{ f ( x ) : x ∈ 𝒳 , ∄ {x}^{′} ∈ 𝒳 s . t . f ( {x}^{′} ) ≻ f ( x ) \} \qquad ( 3 )$$

In some example embodiments, the quality of an approximate Pareto set [symbol] may be evaluated by computing its distance (or proximity) from the optimal Pareto set [symbol] * in the objective space, or d(f( [symbol] ), f( [symbol] *)). The distance metric d: [symbol] × [symbol] → [symbol] may quantify the difference between the sets of objectives, wherein [symbol] denotes the power set of the objective space [symbol] . Although conventional acquisition functions, including improvement-based acquisition functions such as hypervolume improvement (HVI), may be sensitive to any type of improvement (e.g., whenever an approximate set A dominates another approximation set B), those acquisition functions are also sensitive to scaling and transformation of the objective. In fact, the scaling may be super-polynomial with respect to the quantity M of objectives f, which renders conventional improvement-based acquisition functions impractical.

In some cases, a (weak) Pareto-dominance relationship may be used as a preference relationship [symbol] on the search space χ to indicate that a molecule design x is at least as good as another molecule design y (e.g., x [symbol] y) if and only if ∀1≤i≤M:fᵢ(x)≥fᵢ(y). This relationship may be canonically extended to sets of molecule designs where a set A⊆X weakly dominates another set B⊆X (e.g., A [symbol] B) if and only if ∀_{y}∈B∃x∈A: x [symbol] y. Given the preference relationship [symbol] , the goal of multi-objective Bayesian optimization (BO) may include identifying a set of molecule designs that approximates the set of Pareto-optimal solutions and is not strictly dominated by any other sets of approximate Pareto-optimal solutions.

Since the generalized weak Pareto dominance defines a partial order on Ω, there may be incomparable sets in Ω that can cause difficulties with respect to search and utility assessment. These difficulties may be exacerbated at higher values of M. Thus, one way to circumvent this problem may be to define a total order on Ω, which guarantees that any two objective vector sets are mutually comparable. To this end, quality indicators, such as the utility metrics noted above, may be introduced to assign, in the simplest case, each set of approximate Pareto-optimal solutions a real number, such as a unary indicator I that is a function I:Ω→ [symbol] . In some cases, this quality indicator (or utility metric) should exhibit Pareto compliance, which means that it must not contradict the order inducted by the Pareto dominance relationship. Thus, whenever A [symbol] B∧B [symbol] A, the indicator value for A cannot be worse than that of B. A stricter version of Pareto compliance may require that the indicator value of A be strictly better (e.g., higher) than the indicator value of B as indicated in Equation (4) below.

$$A ≽ B ∧ B ⋡ A ⇒ I ( A ) > I ( B ) \qquad ( 4 )$$

In some example embodiments, the cumulative distribution function (CDF) acquisition function α(x) may be applied to determine, each molecule design x, a quality indicator I. In some cases, the cumulative distribution function (CDF) of a real-valued random variable Y may be a function shown in Equation (5) below. According to Equation (5), the cumulative distribution function (CDF) of a real-valued random variable Y may correspond to the probability that the real value of Y is less than or equal to y.

$${F}_{Y} ( y ) = P ( Y \leq y ) = {\int }_{- ∞}^{y} {p}_{Y} ( t ) dt \qquad ( 5 )$$

Where there are an M quantity of objectives, the joint or multivariate cumulative distribution function (CDF) may be given by Equation (6) below.

$${F}_{{Y}_{1} , … , {Y}_{M}} = P ( {Y}_{1} \leq {y}_{1} , … , {Y}_{M} \leq {y}_{m} ) = {\int }_{( - ∞ , … , ∞ )}^{( {y}_{1} , … , {y}_{M} )} {p}_{Y} ( s ) d s \qquad ( 6 )$$

It should be appreciated that every multivariate cumulative distribution function (CDF) may be monotonically non-decreasing for each objective (or constituent variable), right-continuous in each objective (or constituent variable), and 0≤F_{Y}_{₁}_{, . . . , Y}_{_{M}} (y₁, . . . , y_{M})≤1. The monotonically non-decreasing property means that F_{γ}(a₁, . . . , aₘ)≥F_{γ}(b₁, . . . , b_{M}) whenever a₁≥b₁, . . . , a_{K}≥y_{M}. In some cases, these properties may be leveraged when defining various example embodiments of the cumulative distribution function (CDF) indicator described herein.

In some example embodiments, a cumulative distribution function (CDF) indicator I_{F} may be defined, in accordance with Equation (7) below, as the maximum multivariate rank.

$${I}_{FY} ( A ) := {max}_{y ∈ A} {F}_{Y} ( y ) \qquad ( 7 )$$

wherein A denotes an approximation set in Ω. In some cases, the cumulative distribution function (CDF) indicator I_{F} defined in accordance with Equation (7) is compliant with the concept of Pareto dominance. In particular, for any arbitrary approximation sets A∈Ω and B∈Ω, it holds that A [symbol] B∧B [symbol] A⇒I_{F}(A)>I_{F}(B).

In some cases, computing a multivariate joint distribution F_{γ} is a challenging task. For example, a naïve approach may include estimating the multivariate density function before computing its integral, the latter being an especially computationally intensive task. As such, in some example embodiments, copulas (e.g., bivariate copulas) may be used to estimate the cumulative distribution function (CDF) indicator I_{F}. For instance, in some cases, the continuous random vector Y=(Y₁, . . . , Y_{M}) may have a joint distribution F as well as marginal distributions Y₁, . . . , Y_{M} if and only if there exist a unique copula C that is the joint distribution of U=(U₁, . . . , U_{M})=F₁(Y₁), . . . , F_{d}(Y_{M}). Accordingly, a copula may be a multivariate distribution function C: [0,1]^{M}→[0,1] that joints (or couples) uniform marginal distributions in accordance with Equation (8) below. Thus, the multivariate cumulative joint distribution (CDF) may be accessible through computing the copula C. Moreover, it should be appreciated that in order to estimate the copula C, the individual objectives (or molecular properties of interest) may be transformed into uniform marginal distributions by probability integral transform (PIT) of the marginals. In some cases, the probability integral transform (PIT) of an objective Y with the distribution F_{γ} may be the uniformly distributed random variable U=F_{γ}(y) (e.g., U˜Unif([0,1])).

$$F ( {y}_{1} , … , {y}_{M} ) = C ( {F}_{1} ( {y}_{1} ) , … , {F}_{d} ( {y}_{M} ) ) \qquad ( 8 )$$

Using copulas as an estimator for the cumulative distribution function (CDF) indicator may have several advantages. For example, copula-based estimation exhibit scalability and flexible estimation in higher dimensional objective spaces and may be scale invariant with respect to different objectives. Furthermore, copula-based estimation may be invariant under monotonic transformation of the objectives. For instance, letting Y₁ and Y₂ be continuous random variables with copula C_{Y}_{₁}_{,Y}_{₂}, then C_{α(Y}_{₁}_{),β(Y}_{₂}₎=C_{Y}_{₁}_{,Y}_{₂}, y₂ if α, β: [symbol] → [symbol] are strictly increasing functions where C_{α(Y}_{₁}_{),β(Y}_{₂}₎ is the copula function corresponding to the variables α(Y₁) and β(Y₂). These properties of copula-based estimation indicate that the cumulative distribution function (CDF) indicator resulting therefrom may be more robust than the utility metrics derived from conventional improvement based acquisition functions.

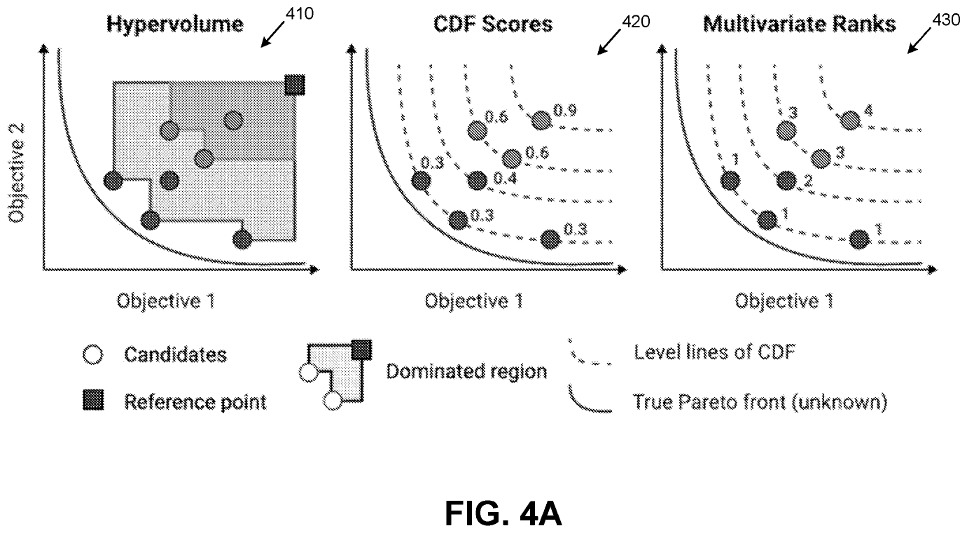

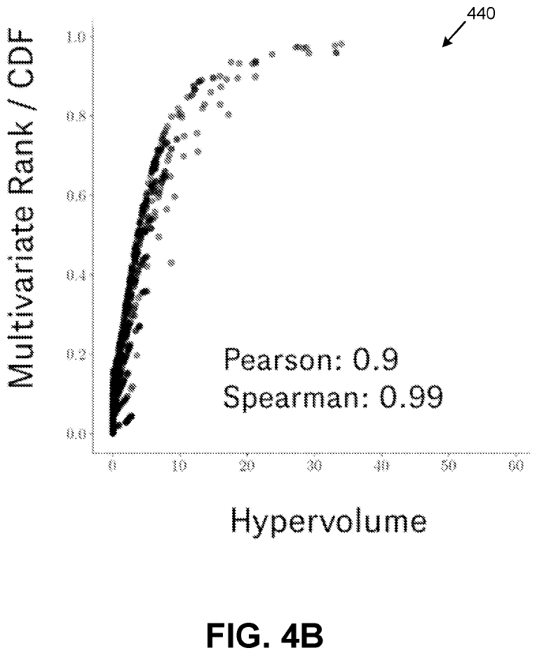

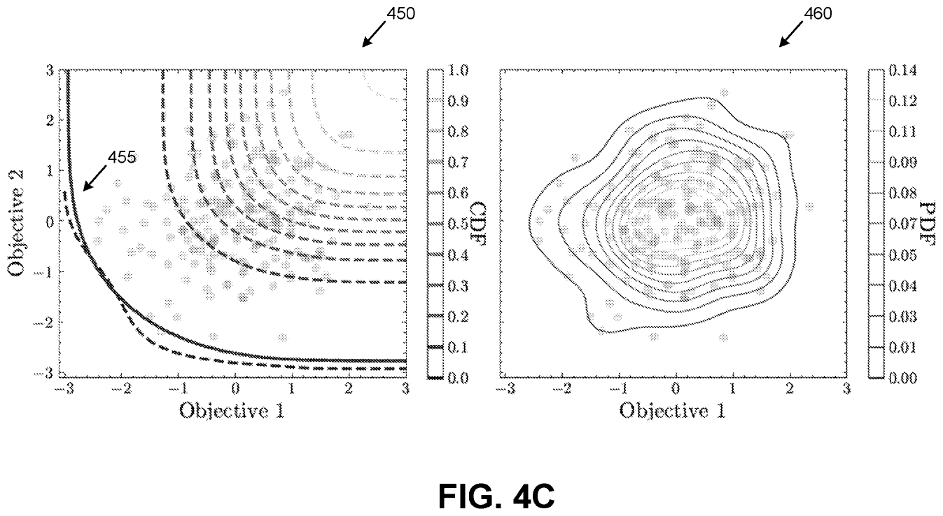

FIG. 4A depicts a schematic diagram illustrating a comparison of different utility metrics including the hypervolume, multivariate ranks, and cumulative distribution function (CDF) indicators of different molecule designs (candidates) being optimized for two objectives, Objective 1 and Objective 2. The hypervolumes (HV) (or polytopes) bounded by each of the candidates are shown in graph 410 while the cumulative distribution function (CDF) indicators and the multivariate ranks of the candidates are shown in graphs 420 and 430, respectively. As shown in FIG. 4A, the cumulative distribution function (CDF) indicators of the molecule designs match that of the multivariate ranks. Moreover, the hypervolumes (or polytopes) bounded by the candidates are also consistent with the multivariate ranks and the cumulative distribution function (CDF) indicators of the molecule designs. This correspondence between the hypervolumes (HV), multivariate ranks, and the cumulative distribution function (CDF) indicators of candidate molecules is further shown in FIG. 4B. Graph 440 in FIG. 4B, which plots the hypervolumes (HV) against the corresponding multivariate ranks and cumulative distribution function (CDF) indicators, show a strong correlation between the utility metrics. The three metrics exhibit a Pearson correlation coefficient of 0.9 and a Spearman correlation coefficient of 0.99. The relationship between multivariate ranking via the cumulative distribution function (CDF) and the corresponding probability density function (PDF) is shown in FIG. 4C. Graph 460 in FIG. 4C shows the probability density function (PDF) that is fit on 200 outcome samples (gray dots) via kernel density estimation (KDE), where the outcome samples are drawn from an elliptical Gaussian distribution Graph 450 shows the level lines of the corresponding cumulative distribution function (CDF). The α level lines converge to approximate the Pareto frontier as α→0. The lowermost level line closely traces the convex shape of the true Pareto frontier 455.

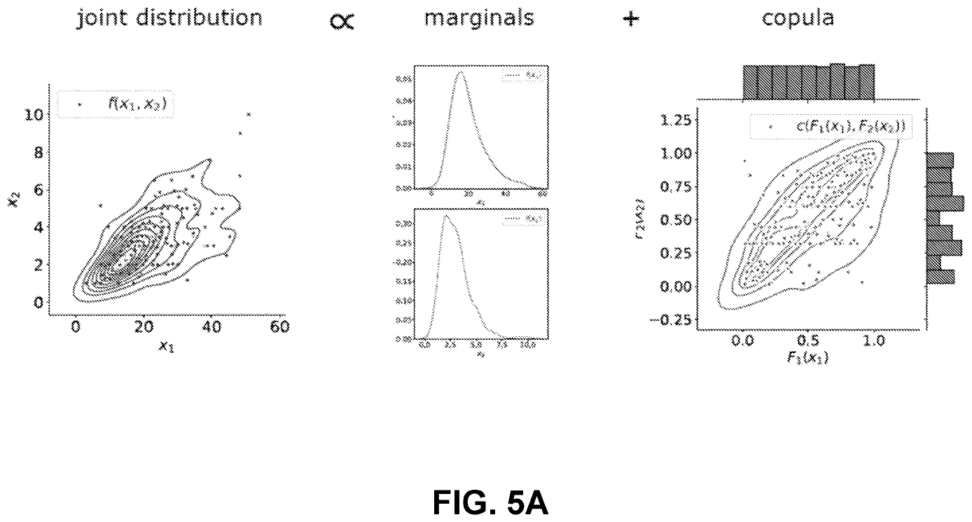

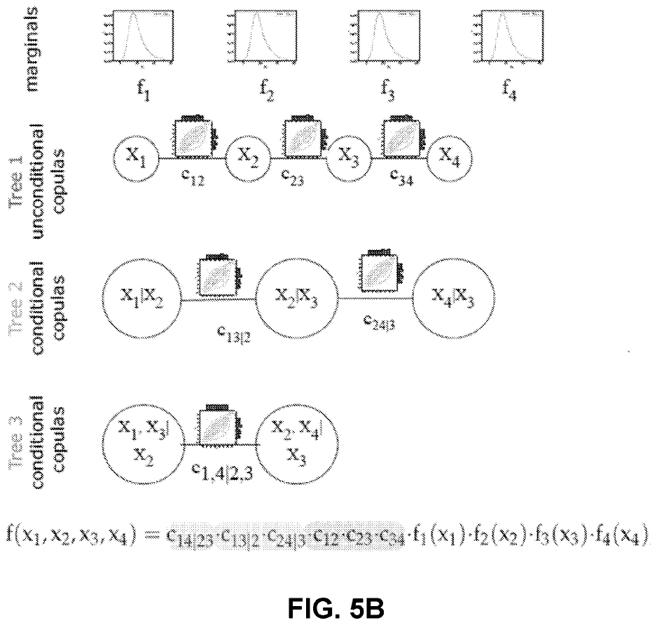

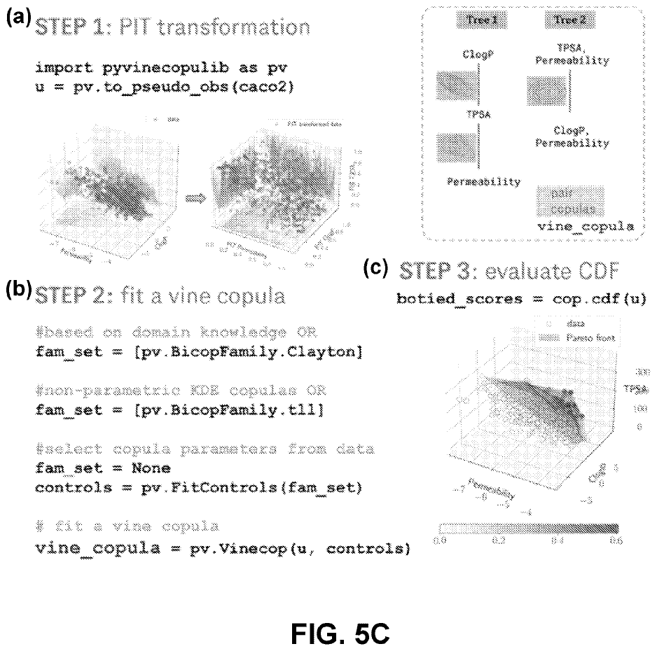

As noted, in some cases, copulas may be used to estimate the cumulative distribution function (CDF) indicator of each computationally generated molecule design. In some cases, the cumulative distribution function (CDF) indicator of a molecule design may correspond to a multivariate rank of the molecule design. As such, in some cases, the cumulative distribution function (CDF) indicator of a molecule design may be estimated by at least decomposing the underlying multivariate joint distribution into two or more marginal distributions of the objectives being optimized (or molecular properties of interest) and one or more copulas (e.g., bivariate copulas). This is illustrated schematically in FIG. 5A where the joint distribution f(x₁, x₂) across two variables x₁ and x₂ is decomposed into a first marginal distribution of the first variable x₁ and a second marginal distribution of the second variable x₂ as well as the copula c(F₁(x₁), F₂(x₂)) describing the inter-correlation therebetween.

In some cases, each copula may be modeled following a parametric family depending on the shape of the dependence structure. Examples of different shaped dependence structures include a Clayton copula, a Gumbel copula, a Gaussian copula, and/or the like. In some cases, pairwise factorization may be performed in order to decompose the multivariate joint distribution into bivariate copulas coupling paired groupings of the marginal distributions of the objectives being optimized (or molecular properties of interest). Doing so may generate a structure (e.g., a graphical model) called a vine that includes an

$$\frac{M ( M - 1 )}{2}$$

quantity of trees in which each edge is representative of a bivariate copula associated with a parametric or non-parametric estimator. Given the foregoing formulation, a cumulative distribution function (CDF) \hat{F}(·; [symbol] ₜ) may be fit on the Nₜ measurements y⁽¹⁾, y⁽²⁾, . . . , y^{(N}^{ₜ}⁾ obtained so far to yield the acquisition function shown as Equation (9) below.

$$u ( x , \hat{f} , {𝒟}_{t} ) = \hat{F} ( \hat{f} ( x ) ; {𝒟}_{t} ) \qquad ( 9 )$$

As noted, in some cases, pairwise factorization may be performed to decompose a multivariate joint distribution into marginal distributions coupled by one or more bivariate copulas (or pair copulas). To further illustrate, it should be appreciated that the joint density of any bivariate random vector (X₁, X₂) may be expressed as Equation (10) below, in which fᵢ are the marginal densities, Fᵢ are the marginal distributions, and c is the copula density.

$$f ( {x}_{1} , {x}_{2} ) = {f}_{1} ( {x}_{1} ) {f}_{2} ( {x}_{2} ) c ( {F}_{1} ( {x}_{1} ) , {F}_{2} ( {x}_{2} ) ) \qquad ( 10 )$$

Accordingly, any bivariate density may be uniquely described by the product of its marginal densities and a copula density, the latter being a dependence structure specifying the inter-correlation between the marginal densities. In cases where there are more than two objectives (or molecular properties of interest), bivariate copula (or pair copula) constructions may be used. The bivariate copulas formed by the decomposition of a joint multivariate distribution having more than two variables may form a vine, which is a hierarchical structure having cascades of bivariate copula blocks. In some cases, any M-dimensional copula density can be decomposed into a product of

$$\frac{M ( M - 1 )}{2}$$

bivariate (conditional) copula densities. Even though the same joint multivariate distribution may be factorized in different ways, the resulting bivariate copulas may form a sequence of M−1 nested trees, which are called vines. A tree may be denoted as Tₘ=(Vₘ, Eₘ) with Vₘ and Eₘ denoting the sets of nodes and edges of the tree m for m=1, . . . , M−1. In this case, each edge e in the set Eₘ of the tree m may be associated with a bivariate copula. An example of a vine having six bivariate copulas resulting from the pairwise factorization of a joint multivariate distribution having four variables x₁, x₂, x₃, and x₄ and four marginal distributions f₁, f₂, f₃, and f₄ is shown in FIG. 5B. In practice, a single vine may include two components. First is the structure of the vine, which includes the set of trees Tₘ=(Vₘ, Eₘ) for m∈[M−1]. Second are the bivariate copulas (or pair-copulas) cⱼ_{ₑ}_{,k}_{ₑ}_{|D}_{ₑ} for e∈Eₘ and m∈[M−1].

In a low-data regime where few molecule designs with actual wet lab measurements are available, the empirical Pareto frontier may be especially noisy. As such, in cases, domain knowledge regarding one or more of the objectives being optimized (or molecular properties of interest), such as the relative priorities of at least some objectives, may be leveraged when using vine copulas to construct a model-based Pareto frontier. For example, in some cases, known correlations among the objectives (or molecular properties of interest) may be incorporated to specify the hierarchical structure of the vine. Moreover, the choice of copula models (e.g., the type of dependence including tail behavior) may be informed by the pairwise joint distributions, which may be approximated based on domain knowledge. As such, it should be appreciated that the advantages of integrating copula-based estimators in the multivariate rank based utility metric and acquisition function described herein includes scalability from the convenient pair copula construction of vines, robustness with respect to marginal scales and transformations due to inherent copula properties, and domain-aware copula structures from explicit encoding of dependencies in the vine copula matrix, including the choice of dependence type (e.g., low or high tail dependence).

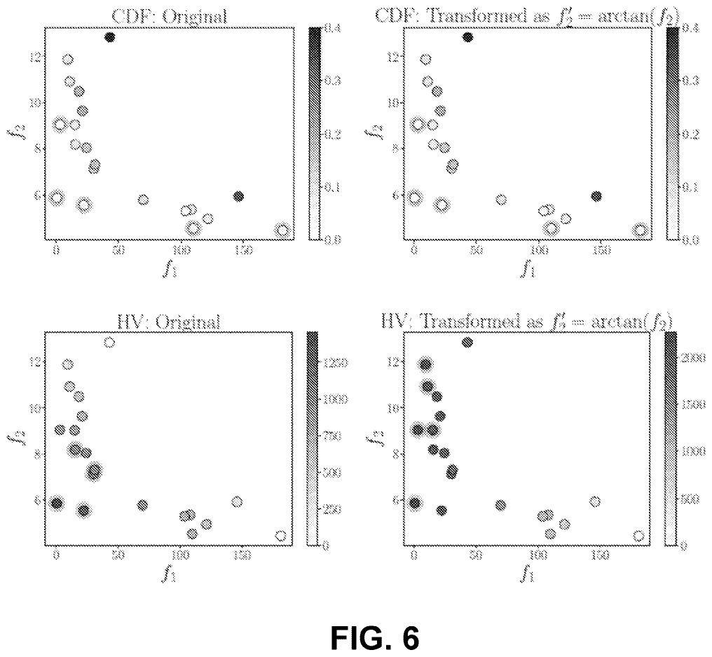

To further illustrate, FIG. 5C depicts the use of copulas in the context of optimizing multiple objectives in the drug discovery, where data tends to be sparse. Panel (a) shows a probability integration transformation (PIT) to yield uniform margins before a vine copula is fit in panel (b). The fitting of the vine copula in panel (b) may include the selection of a copula shape, for example, from parametric or non-parametric families. Finally, panel (c) shows the evaluation of the cumulative distribution function (CDF) from the copula. It should be appreciated that due to the separate estimation of marginal distribution and copulas (e.g., dependence structures), different marginal distributions may have the same Pareto front in the probability integral transform (PIT) space in which the cumulative distribution function (CDF) indicators are evaluated. As such, with copula based estimators, the resulting cumulative distribution function (CDF) indicators may be robust without any overhead for scalarization or standardization (e.g., across different units of measurement), as is required for conventional acquisition functions and utility metrics. That is, regardless of the distributions of the marginals, the cumulative distribution function (CDF) indicator estimated using copulas remains the same. This robustness to arbitrary scaling of the objectives is further shown in the graphs in FIG. 6. As shown in FIG. 6, the values of the conventional hypervolume (HV) indicator are sensitive to scaling (e.g., f₂ being transformed to

$${f}_{2}^{′}$$

via arctan(f₂)) whereas the values of the cumulative distribution function (CDF) indicator are scale-invariant.

Panel (b) shows that domain knowledge, including the interplay between different objectives (or molecular properties of interest), may be encoded in the hierarchical structure of the copula based estimator, in this case for the Caco2+ dataset associated with the Caco-2 cell line derived from human colorectal adenocarcinoma cells. In the Caco2+ example shown in Panel (b), the permeability of a molecule design is often highly positively correlated with its lipophilicity (measured by computed log P (Clog P)) and negatively correlated with its topological polar surface area (tPSA). These correlations are especially notable at the tails of the data distribution. Accordingly, in some cases, such dependencies may be encoded in the vine copula structure and in the choice of copula family for each pair. For instance, for the Caco2+ example, a rotated Clayton copula may be selected to preserve the tail dependence between the topological polar surface area (tPSA) and the permeability of molecule designs.

Table 1 below depicts an example of the algorithm for multi-objective Bayesian optimization (MOBO) with a multivariate rank based acquisition function that uses copulas to estimate a cumulative distribution function (CDF) indicator corresponding to the multivariate rank of each molecule design.

TABLE 1
Algorithm 1: MOBO with a multivariate rank based acquisition function

```
  $1 : Input : Probabilistic surrogate \hat{f} , initial data {𝒟}_{0} = {\{ ( {x}_{n} , {y}_{n} ) \}}_{n = 1}^{{N}_{0}} , 𝒳 ⊂ {ℝ}^{d} , 𝒴 ⊂ {ℝ}^{M}$
  2: Output: Optimal selected subset [symbol] _{T}
  3: Fit the initial surrogate model \hat{f} (xᵢ) on [symbol] ₀
  4: for {t = 1, ... , T} do
  5: Sample the candidate pool x₁, ... , x_{N} ∈ [symbol]
  6: for {i = 1, ... , N} do
  7: Evaluate \hat{f} o the candidate pool to obtain the posterior p(f(xᵢ)| [symbol] ₜ₋₁)
  $8 : Draw L predictive samples {\hat{f}}_{i}^{( j )} ∼ p ( f ( {x}_{i} ) \vert {𝒟}_{t - 1} ) , for j ∈ [ L ]$
  9: end for
$10 : Obtain uniform marginals {\{ {u}_{i}^{( j )} \}}_{i ∈ [ N ] , j ∈ [ L ]} from the pooled samples {\{ {\hat{f}}_{i}^{( j )} \}}_{i ∈ [ N ] , j ∈ [ L ]}$
11: Version 1: Fit a vine copula Ĉ on the uniform marginals on the sample level,
      ${\{ {u}_{i}^{( j )} \}}_{i ∈ [ N ] , j ∈ [ L ]}$
      Version 2: Fit a vine copula Ĉ on the mean-aggregated uniform marginals,
      ${\{ \frac{1}{L} {\sum }_{j = 1}^{L} {u}_{i}^{( j )} \}}_{i ∈ [ N ]}$
12: for {i = 1, ... , N} do
$13 : Version 1 : Compute the expected CDF score ( {x}_{i} ) = \frac{1}{L} {\sum }_{j = 1}^{L} \hat{C} ( {u}_{i}^{( j )} )$
        $Version 2 : Compute the CDF score of the mean ranks ( {x}_{i} ) = \hat{C} ( \frac{1}{L} {\sum }_{j = 1}^{L} {u}_{i}^{( j )} )$
14: end for
$15 : {i}^{*} ← arg {max}_{i ∈ [ N ]} ( {x}_{i} )$
16: [symbol]  ₜ ← [symbol] ₜ₋₁ ∪ {(x*, y*)}
17: end for
18: return [symbol] _{T}
```

As noted, in a scenario, such as drug design, with M objectives (or molecular properties of interest), the multivariate rank of a molecule design x may correspond to a cumulative distribution function (CDF) indicator estimated using one or more copulas (e.g., bivariate copulas). For example, in some cases, at least an M quantity of property computation models fₘ: [symbol] → [symbol] for m=1, . . . , M may be applied, each of which being a probabilistic surrogate model approximating a probability distribution enumerating the probability of occurrence of each possible value of a corresponding objective (or molecular property of interest). In some cases, a single molecule design x with the best values for every objective (or molecular property of interest) may not exist due to the competing nature of at least some objectives (or molecular properties of interest). As such, the goal of multi-objective Bayesian optimization (MOBO) may be to identify the set of Pareto-optimal solutions, which are molecule designs x in which improving one objective (or molecular property of interest) leads to the worsening of at least one other objective (or molecular property of interest). For instance, the Pareto-optimal solutions for optimization across expression level and binding affinity may be a set of molecule designs x in which an improvement in expression level is accompanied by a decrease in binding affinity. In some cases, a molecule design x that is a Pareto-optimal solution may be identified based on its utility metric, which in this case is a cumulative distribution function (CDF) indicator corresponding to the multivariate rank of the molecule design. The cumulative distribution function (CDF) indicator of two molecule designs x₁ and x₂ may order the molecule designs x₁ and x₂ based on their respective quality as Pareto-optimal solutions. The first molecule design x₁ having a better cumulative distribution function (CDF) indicator than the second molecule design x₂ may indicate that the first molecule design x₁ is more likely to be a Pareto-optimal solution exhibiting a better combination of properties (e.g., drug-like properties) than the second molecule design x₂. In this case, the first molecule design x₁ may be said to dominate the second molecule design x₂ while being more proximate to the Pareto frontier populated by Pareto-optimal (or non-dominated) solutions.

In some cases, sequential optimization, which includes querying the property computation models f for a single molecule design per design iteration, may be impractical for many applications due to the latency in feedback. In protein engineering, for example, it may be necessary to select a batch of molecule designs in a given iteration and wait several months to receive measurements. Jointly selecting a batch of q molecule designs from a large pool of q′>>q candidates may require combinatorial evaluations of the acquisition function which, in some cases, may be a multivariate rank based acquisition function and not a conventional acquisition function like an improvement-based acquisition function or an entropy based acquisition function. In the context of optimizing molecule designs based on the gradient of the acquisition function, sequential greedy selection of q molecule designs during each design iteration may achieve comparable performance relative to the joint selection of q candidates for a variety of acquisition functions including the multivariate rank based acquisition function described herein.

Many molecule design applications require the enforcement of some hierarchy amongst multiple objectives (or molecular properties of interest). For example, in some cases, a partial ordering of M objectives (or molecular properties of interest) in which some properties are prioritized over others may be expressed as ordered sets of properties: {y_{0,0}, . . . , y_{0,M}_{₀}}→{y_{1,0}, . . . , y_{1,M}_{₁}}→ . . . →{y_{L,0}, . . . , y_{L,M}_{_{L}}}, wherein y_{l,m} denotes the property at level l∈{0, . . . ,-−1} of the hierarchy and m∈{0, . . . , Mₗ₋−1} is its index among the Mₗ sibling properties at the same level 1. As noted, in some cases, the partial ordering may arise from an experimental dependency in which one property of a molecule design must satisfy one or more criteria (e.g., pass a certain threshold) before its other properties can be measured. Alternatively and/or additionally, the partial ordering may reflect a preference for certain types of molecule designs. For example, in some cases, one or more properties may be prioritized such that molecule designs that perform poorly in these properties may be rejected no matter how well the molecule designs perform in other properties. In some cases, a partial ordering of M objectives (or molecular properties of interest) may be encoded when performing the pairwise factorization of the corresponding M-dimensional joint distribution. The pairwise factorization of the M-dimensional joint distribution may generate a vine, which is a hierarchical structure having a sequence of M−1 nested trees linked by an

$$\frac{M ( M - 1 )}{2}$$

number of bivariate copulas. In some cases, the hierarchical structure of the vine may correspond to the partial ordering of the M objectives (or molecular properties of interest) in which a first objective that is prioritized over a second objective may occupy a higher level of the hierarchical structure than the second objective.

### Example Tasks for Multi-Objective Bayesian Optimization (MOBO)

The performance of the selection engine 120 using various different acquisition functions and utility metrics was evaluated through experiments on four tasks, including the simulated tasks Branin-Currin, DTLZ, and penicillin production, and a real-world drug design set up in the form of Caco2+. For these example use cases, the selection engine 120 performed multi-objective Bayesian optimization (MOBO) using a hypervolume (HV) indicator and the cumulative distribution function (CDF) indicator described herein. The different acquisition functions including conventional acquisition functions such as noisy expected hypervolume improvement (NEHVI) acquisition function, noisy Pareto efficient global optimization (noisy ParEGO) acquisition function, and two versions of the multivariate rank based acquisition function described herein. The Bayesian optimization (BO) simulation was batched with a batch size of B=4 for all experiments while the number T of design iterations varied. Other parameters for the experiments included the initial data size No, the size of the design candidate pool N, and the number of predictive posterior samples L output by each property computation model. The size of the pool was fixed relative to the selected batch at

$$\frac{N}{B} = 100$$

and the number of predictive posterior samples is fixed to L=20.

For the DTLZ benchmark task, the selection engine 120 performed T=30 design iterations with d=9 and three different numbers of objectives M∈{4,6,8}. The Penicillin production problem include T=10 design iterations for d=7 and M=3. The Branin-Currin benchmark task (d=2, M=2) is a composition of the Branin and Currin functions featuring a concave Pareto front (in the maximization setting) that includes maximizing the two functions f₁ and f₂ of the two objectives x₁ and x₂ as shown below. The selection engine 120 perform multi-objective Bayesian optimization (MOBO) over T=30 design iterations.

$${f}_{1} ( {x}_{1} , {x}_{2} ) = - {( {x}_{2} - \frac{5.1}{4 {π}^{2}} {x}_{1}^{2} + \frac{5}{π} {x}_{1} - r )}^{2} + 10 ( 1 - \frac{1}{8 π} ) cos ( {x}_{1} ) + 10$$

$${f}_{2} ( {x}_{1} , {x}_{2} ) = - [ 1 - exp ( \frac{1}{2 {x}_{2}} ) ] \frac{2300 {x}_{1}^{3} + 1900 {x}_{1}^{2} + 2092 {x}_{1} + 60}{100 {x}_{1}^{3} + 500 {x}_{1}^{2} + 4 {x}_{1} + 20}$$

wherein x₁, x₂∈[0,1].

Returning to the Caco2+ example, this task was performed for T=10 design iterations to optimized M=3 objectives (or molecular properties of interest). The Caco2+ example uses the Caco2+ dataset generated by modifying the Caco-2 dataset, which includes 906 drug molecules annotated with experimentally measured rates of permeability through a human colon epithelial cancer cell line. Each of these molecules is represented as a concatenation of fingerprint and fragment feature vectors. The modifications include augmenting the dataset with additonal properties such as a drug-likeness score (Quantitative Estimate of Druglikeness (QED)) and topological polar surface area (TPSA). As described in more details below, subsets of these properties, such as permeability and topological polar surface area (TPSA), may be competitive, meaning improving one property will lead to the worsening of the other. Such tradeoffs become more dramatic as additional objectives (or molecular properties of interest) are introduced, as is often the case during late-state drug optimization.

As noted, in the case of the Caco2+ task, the goal of the multi-objective Bayesian optimization is to identify molecule designs exhibiting maximum cell permeability, which is a measure of the degree to which a molecule passes through a cellular membrane. Permeability is often a critical property in drug discovery (DD) programs where the disease protein being targeted is intracellular, meaning that it resides within cells. For this experiment, a molecule design x₁ is applied to a monolayer of Caco2 cells and, after incubation, the concentration c of xᵢ is measured on both the input and output side of the monolayer to give the values cᵢₙ and cₒᵤₜ. The ratio

$$\frac{{c}_{in}}{{c}_{out}}$$

is treated as the final permeability label

$${y}_{i}^{p}$$

of the molecule design x₁.

Cellular membranes include a complex mixture of lipids and other biomolecules. In order to enter and (passively) diffuse through a membrane, a molecule should interact favorably with these biomolecules and/or avoid disrupting their packing structure. Increasing the lipophilicity (log P) of the molecule design xᵢ is therefore one strategy for increasing permeability

$${y}_{i}^{p} .$$

However, increasing lipophilicity (log P) also increases the promiscuous binding of the molecule to non-disease related proteins, which can lead to undesired side effects. As such, in some cases, the multi-objective Bayesian optimization (MOBO) task may include optimizing two competing objectives by at least reducing (or minimizing) the computed log P

$$( clogP , {y}_{i}^{l} )$$

of the molecule design xᵢ while increasing (or maximizing) its permeability

$${y}_{i}^{p} .$$

In addition, other objectives for multi-objective Bayesian optimization in drug discovery (DD) settings may include increasing the affinity and specificity of binding towards a target molecule. As opposed to the aforementioned non-specific lipophilic interaction, polar contacts (e.g., hydrogen bonds) between drug molecules and the target protein molecules may result in higher affinity and more specific binding. The topological polar surface area

$$( TPSA , {y}_{i}^{t} )$$

of a molecule design xᵢ is one indicator of its ability to form such interactions and is therefore another objective that is optimized for the Caco2+ task. However, as is the case with decreasing log P, increasing the topological polar surface area

$$( TPSA , {y}_{i}^{t} )$$

of a molecule design xᵢ can negatively impact its permeability

$${y}_{i}^{p} .$$

As such, the topological polar surface area

$$( TPSA , {y}_{i}^{t} )$$

of a molecule design xᵢ and its permeability

$${y}_{i}^{p}$$

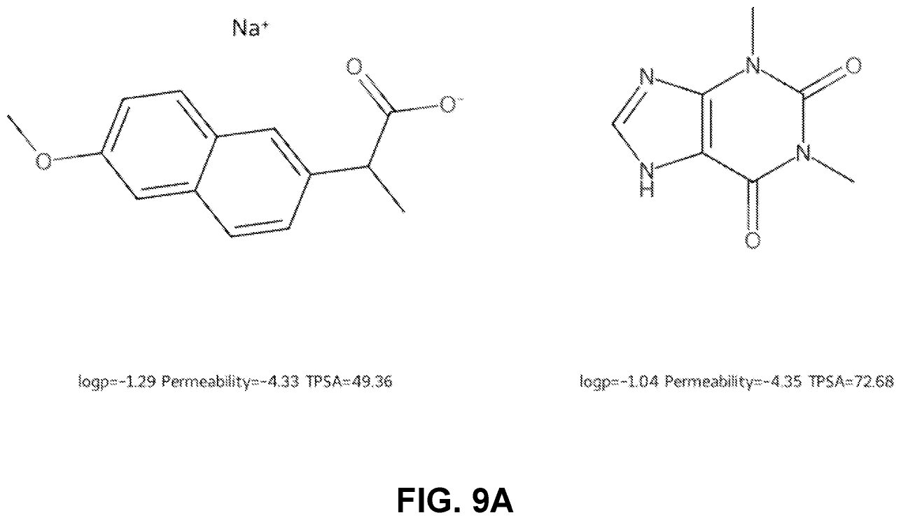

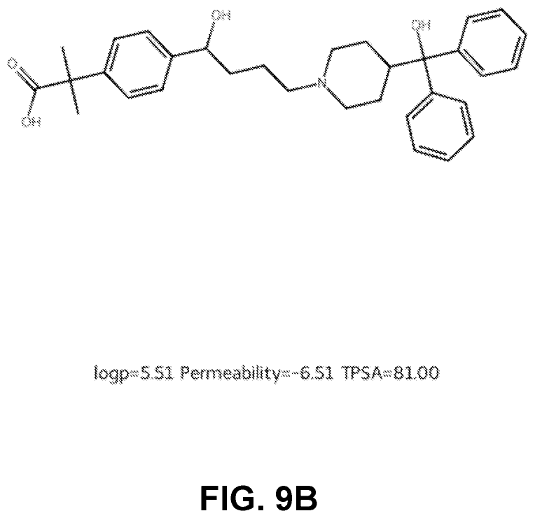

are also competing objectives. To further illustrate, FIG. 9A depicts two examples of molecule designs with a desirable combination of topological polar surface area (TPSA), permeability, and lipophilicity (computed log P) values. Contrastingly, an example of a molecule design with a poor combination of topological polar surface area (TPSA), permeability, and lipophilicity (computed log P) values is shown in FIG. 9B.

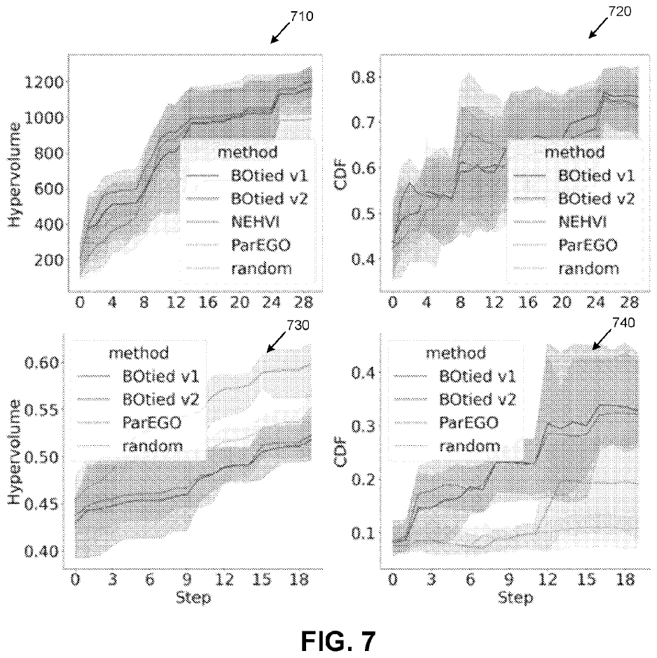

In view of these experimental tasks, the tables in FIG. 7 depict the changes in the values of different utility metrics over the course of multiple rounds of multi-objective Bayesian optimization (MOBO). Referring to FIG. 7, the changes in values of the hypervolume (HV) indicator and the cumulative distribution function (CDF) indicator of molecule designs generated for the Branin-Currin task over 30 rounds of multi-objective Bayesian optimization (MOBO) are shown in graphs 710 and 720. The changes in the values of the hypervolume (HV) indicator and the cumulative distribution function (CDF) indicator of molecule designs generated for the DTLZ task over 30 rounds of multi-objective Bayesian optimization (MOBO) are shown in graphs 710 and 720.

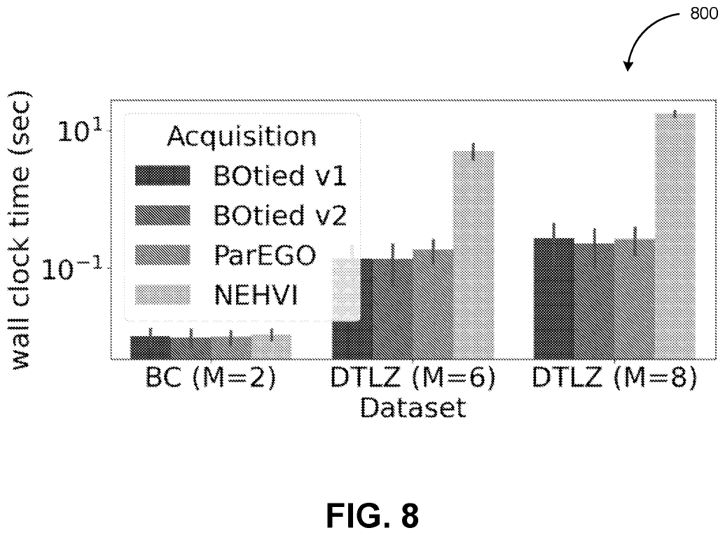

For a further comparison of the hypervolume (HV) indicator and the cumulative distribution function (CDF) indicator, Table 2 below shows the mean and standard deviation of the values of the hypervolume (HV) indicator, computed in original units, and the values of the cumulative distribution function (CDF) indicator for different tasks. It should be appreciated that higher means and standard deviations are considered better. The two versions of the multivariate ranking based acquisition function described herein are annotated as MVR v1 and MVR v2. As shown in Table 2, the performance of noisy expected hypervolume improvement (NEHVI) degrades with higher values of M. Meanwhile, graph 800 in FIG. 8 compares the wall clock time per single call of the different acquisition functions for the Branin-Currin and DTLZ tasks. As shown in FIG. 8, while the multivariate ranking based acquisition function described herein achieved comparable performance when it comes to identifying Pareto optimal solutions across different tasks, it does so faster due to the reduced computational complexity of using copulas to estimate cumulative distribution function (CDF) indicators. Furthermore, it should be appreciated that the cumulative distribution function (CDF) indicator described herein have a more interpretable scale than hypervolume indicators, whose scales do not carry any information about the internal ordering of different molecule designs. For example, in some cases, the cumulative distribution function (CDF) indicator described herein may be bounded in value between 0 and 1, with values closer to 1 being associated with Pareto-optimal solutions that are proximate to the Pareto frontier.

TABLE 2

| | BC (M = 2) | | | DTLZ (M = 4) | | | | DTLZ (M = 6) | | DTLZ (M = 8) | | |
|---|---|---|---|---|---|---|---|---|---|---|---|---|
| | CDF | HV | | CDF | | HV | | CDF | HV | CDF | | HV |
| MVR v1 | 0.76 (0.06) | 1164.43 | (174.37) | 0.2 | (0.1) | 0.42 | (0.03) | 0.33 (0.09) | 0.52 (0.02) | 0.2 | (0.08) | 0.93 (0.02) |
| MVR v2 | 0.74 (0.08) | 1205.3 | (120.46) | 0.24 | (0.2) | 0.45 | (0.05) | 0.32 (0.08) | 0.58 (0.03) | 0.19 | (0.1) | 0.91 (0.03) |
| NParEGO | 0.73 (0.09) | 993.31 | (178.16) | 0.20 | (0.07) | 0.4 | (0.03) | 0.29 (0.03) | 0.69 (0.02) | 0.13 | (0.07) | 1.05 (0.02) |
| NEHVI | 0.73 (0.07) | 1196.37 | (98.72) | 0.21 | (0.02) | 0.44 | (0.04) | — | — | — | | — |
| Random | 0.71 (0.11) | 1204.99 | (69.34) | 0.1 | (0.05) | 0.22 | (0.03) | 0.10 (0.03) | 0.55 (0.02) | 0.13 | (0.07) | 0.96 (0.02) |

| | Caco2+ (M = 3) | | | Penicillin (M = 3) | | | CopulaBC (M = 2) | | | |
|---|---|---|---|---|---|---|---|---|---|---|
| | CDF | HV | | CDF | HV | | CDF | | HV | |
| MVR v1 | 0.58 (0.06) | 11645.63 | (629.0) | 0.48 (0.02) | 319668.6 | (17806.2) | 0.9 | (0.03) | 1.08 | (0.03) |
| MVR v2 | 0.60 (0.06) | 11208.57 | (882.21) | 0.49 (0.02) | 318687.7 | (17906.2) | 0.9 | (0.01) | 1.09 | (0.02) |
| NParEGO | 0.56 (0.05) | 12716.2 | (670.12) | 0.28 (0.09) | 332203.6 | (15701.52) | 0.87 | (0.01) | 1.1 | (0.01) |
| NEHVI | 0.54 (0.06) | 13224.7 | (274.6) | 0.24 (0.05) | 318748.9 | (2868.64) | 0.88 | (0.02) | 1.1 | (0.01) |
| Random | 0.57 (0.07) | 11425.6 | (882.4) | 0.32 (0.02) | 327327.9 | (17036) | 0.88 | (0.02) | 1.08 | (0.01) |

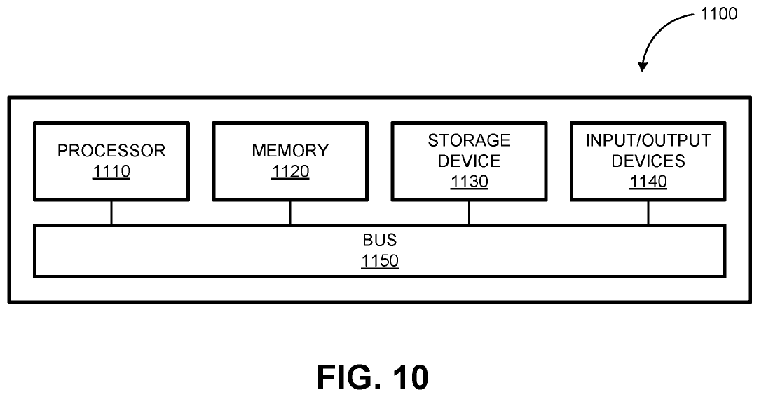

FIG. 10 depicts a block diagram illustrating an example of a computing system 1100, in accordance with some example embodiments. Referring to FIGS. 1-10, the computing system 1100 may be used to implement the molecule design engine 110, the selection engine 120, the laboratory equipment 130, the client device 140, and/or any components therein.

As shown in FIG. 10, the computing system 1100 can include a processor 1110, a memory 1120, a storage device 1130, and input/output devices 1140. The processor 1110, the memory 1120, the storage device 1130, and the input/output devices 1140 can be interconnected via a system bus 1150. The processor 1110 is capable of processing instructions for execution within the computing system 1100. Such executed instructions can implement one or more components of, for example, the molecule design engine 110, the selection engine 120, the laboratory equipment 130, the client device 140, and/or the like. In some example embodiments, the processor 1110 can be a single-threaded processor. Alternately, the processor 1110 can be a multi-threaded processor. The processor 1110 is capable of processing instructions stored in the memory 1120 and/or on the storage device 1130 to display graphical information for a user interface provided via the input/output device 1140.

The memory 1120 is a computer readable medium such as volatile or non-volatile that stores information within the computing system 1100. The memory 1120 can store data structures representing configuration object databases, for example. The storage device 1130 is capable of providing persistent storage for the computing system 1100. The storage device 1130 can be a floppy disk device, a hard disk device, an optical disk device, or a tape device, or other suitable persistent storage means. The input/output device 1140 provides input/output operations for the computing system 1100. In some example embodiments, the input/output device 1140 includes a keyboard and/or pointing device. In various implementations, the input/output device 1140 includes a display unit for displaying graphical user interfaces.

According to some example embodiments, the input/output device 1140 can provide input/output operations for a network device. For example, the input/output device 1140 can include Ethernet ports or other networking ports to communicate with one or more wired and/or wireless networks (e.g., a local area network (LAN), a wide area network (WAN), the Internet).

In some example embodiments, the computing system 1100 can be used to execute various interactive computer software applications that can be used for organization, analysis and/or storage of data in various formats. Alternatively, the computing system 1100 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1140. The user interface can be generated and presented to a user by the computing system 1100 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A system, comprising:
at least one data processor; and
at least one memory storing instructions, which when executed by the at least one data processor, result in operations comprising:
generating a plurality of molecule designs;
applying one or more property computation models to generate an output indicative of a plurality of properties exhibited by the plurality of molecule designs, where the one or more property computation models are trained to approximate a probability distribution of each property of the plurality of properties;
determining, based at least on the output of the one or more property computation models, a cumulative distribution function (CDF) indicator for each molecule design of the plurality of molecule designs, where the cumulative distribution function (CDF) indicator of a molecule design corresponds to a multivariate rank of the molecule design, and where the multivariate rank of the molecule design quantifies a probability that none of the plurality of properties present in the molecule design can be improved without degrading at least one other property of the plurality of properties; and
selecting, based at least on the cumulative distribution function (CDF) indicator of each molecule design, one or more molecule designs from the plurality of molecule designs as candidates for wet lab assessment.

2. The system of claim 1, wherein the one or more property computation models are trained to approximate a probability distribution of a plurality of possible values of a first property of the plurality of properties, and wherein the one or more property computation models are further trained to approximate a probability distribution of a plurality of possible values of a second property of the plurality of properties.

3. The system of claim 2, wherein the output of the one or more property computation models includes a plurality of predictive samples from the probability distribution of the first property and the probability distribution of the second property, and wherein each predictive sample includes a value of the first property and a value of the second property present in the molecule design.

4. The system of claim 1, wherein the cumulative distribution function (CDF) indicator of each molecule design is determined by at least
determining, based at least on the output of the one or more property computation models, a marginal distribution of each property of the plurality of properties,
determining, based at least on the output of the one or more property computation models, one or more copulas describing an inter-correlation between the plurality of properties, and
determining, based at least on the marginal distribution of each property and the one or more copulas, the cumulative distribution function (CDF) indicator of each molecule design.

5. The system of claim 1, wherein the cumulative distribution function (CDF) indicator of each molecule design is determined by at least
determining, based at least on the output of the one or more property computation models, a marginal distribution of a first property of the plurality of properties,
determining, based at least on the output of the one or more property computation models, a marginal distribution of a second property of the plurality of properties, and
determining, based at least on the output of the one or more property computation models, a copula coupling the marginal distribution of the first property and the marginal distribution of the second property by at least describing a dependence between the first property and the second property.

6. The system of claim 5, wherein the cumulative distribution function (CDF) indicator of each molecule design is further determined by at least
determining a marginal distribution of a third property of the plurality of properties, and
determining an additional copula coupling the marginal distribution of the third property and at least one of the marginal distribution of the first property and the marginal distribution of the second property.

7. The system of claim 6, wherein the copula and the additional copula comprise bivariate copulas forming a vine.

8. The system of claim 7, wherein the vine is determined to exhibit a hierarchical structure corresponding to a partial ordering in which the first property is prioritized over the second property and/or the third property.

9. The system of claim 1, wherein the cumulative distribution function (CDF) indicator of each molecule design is determined by at least
performing a pairwise factorization of a multivariate joint distribution corresponding to the plurality of properties to determine one or more pairwise groupings of the plurality of properties, where each pairwise grouping of the plurality of properties corresponds to a bivariate joint distribution, and
determining a bivariate copula coupling each pairwise grouping of the plurality of properties.

10. The system of claim 9, wherein the cumulative distribution function (CDF) indicator of each molecule design is determined by at least
determining, based at least on a tail behavior of the bivariate joint distribution, a type of the bivariate copula coupling each pairwise grouping of the plurality of properties.

11. The system of claim 10, wherein the type of bivariate copula is one of a Clayton copula, a Gumbel copula, or a Gaussian copula.

12. The system of claim 1, wherein the cumulative distribution function (CDF) indicator of each molecule design is determined by at least
determining, based at least on the measurement set, a mean and a covariance of the plurality of properties,
determining, based at least on the mean and the covariance of the plurality of properties, a multivariate Gaussian distribution of a plurality of possible values of the plurality of properties, and
determining, based at least on the multivariate Gaussian distribution, the cumulative distribution function (CDF) indicator of each molecule design.

13. The system of claim 1, wherein the cumulative distribution function (CDF) indicator of each molecule design is determined by at least determining, based at least on the measurement set, an empirical cumulative distribution function, where the empirical cumulative distribution function comprises a step function that increases by 1/n for each of an n quantity of datapoints in the measurement set, and where the empirical cumulative distribution function outputs, for any specified value of the plurality of properties, a value corresponding to a fraction of measurements in the measurement set that are less than or equal to the specified value, and
determining, based at least on the empirical cumulative distribution function, the cumulative distribution function (CDF) indicator of each molecule design.

14. The system of claim 1, wherein the cumulative distribution function (CDF) indicator of each molecule design is determined by at least
performing a kernel density estimation (KDE) to estimate a multivariate joint distribution corresponding to the plurality of properties, and
determining, based at least on an estimate of the multivariate joint distribution, the cumulative distribution function (CDF) indicator of each molecule design.

15. The system of claim 1, wherein the cumulative distribution function (CDF) indicator of each molecule design is an expected cumulative distribution function (CDF) indicator whose value is determined to account for an uncertainty in the output of the one or more property computation models.

16. The system of claim 1, further comprising:
selecting the one or more molecule designs as candidates for wet lab assessment based at least on the one or more molecule designs having a better cumulative distribution function (CDF) indicator than one or more molecule designs generated during a previous iteration of multi-objective Bayesian optimization.

17. The system of claim 1, further comprising:
selecting a molecule design as a candidate for wet lab assessment based at least on a cumulative distribution function (CDF) indicator of the molecule design satisfying one or more thresholds; and
excluding a different molecule design from being a candidate for wet lab assessment based at least on a cumulative distribution function (CDF) indicator of the different molecule design failing to satisfy the one or more thresholds.

18. The system of claim 1, further comprising:
selecting one molecule design instead of another molecule design as a candidate for wet lab assessment based at least on a cumulative distribution function (CDF) indicator of the one molecule design being better than a cumulative distribution function (CDF) indicator of the other molecule design.

19. The system of claim 1, further comprising:
selecting, based at least on the cumulative distribution function (CDF) indicator of each molecule design, a threshold quantity of molecule designs having a best cumulative distribution function (CDF) indicator as candidates for wet lab assessment.

20. The system of claim 1, further comprising:
receiving a measurement set associated with a plurality of prior molecule designs, where the measurement set includes, for each prior molecule design, one or more measurements of a plurality of properties exhibited by each prior molecule design; and
training, based at least on the measurement set, one or more property computation models to approximate the probability distribution of each property of the plurality of properties.

21. The system of claim 19, further comprising:
receiving, for the one or more molecule designs selected as candidates for wet lab assessment, one or more additional measurements for the plurality of properties;
retraining, based at least on the one or more additional measurements, the one or more property computation models; and
applying the one or more retrained property computation models to determine the plurality of properties exhibited by one or more subsequent molecule designs.

22. The system of claim 21, wherein the retraining of the one or more property computation models includes updating, based at least on the one or more additional measurements, the probability distribution of each property of the plurality of properties being approximated by the one or more property computation models.

23. The system of claim 21,
wherein the plurality of prior molecule designs are generated during a previous iteration of multi-objective Bayesian optimization (MOBO), wherein the plurality of molecule designs are generated during a current design iteration of multi-objective Bayesian optimization (MOBO), and wherein the one or more subsequent molecule designs are generated during a subsequent iteration of multi-objective Bayesian optimization (MOBO).

24. The system of claim 1, wherein the one or more property computation models includes at least one ensemble of property computation models in which multiple property computation models are trained to determine a single property of the plurality of properties.

25. A computer-implemented, comprising:
generating a plurality of molecule designs;
applying one or more property computation models to generate an output indicative of a plurality of properties exhibited by the plurality of molecule designs, where the one or more property computation models are trained to approximate a probability distribution of each property of the plurality of properties;
determining, based at least on the output of the one or more property computation models, a cumulative distribution function (CDF) indicator for each molecule design of the plurality of molecule designs, where the cumulative distribution function (CDF) indicator of a molecule design corresponds to a multivariate rank of the molecule design, and where the multivariate rank of the molecule design quantifies a probability that none of the plurality of properties present in the molecule design can be improved without degrading at least one other property of the plurality of properties; and
selecting, based at least on the cumulative distribution function (CDF) indicator of each molecule design, one or more molecule designs from the plurality of molecule designs as candidates for wet lab assessment.

26. A non-transitory computer readable medium storing instructions, which when executed by at least one data processor, result in operations comprising:
generating a plurality of molecule designs;
applying one or more property computation models to generate an output indicative of a plurality of properties exhibited by the plurality of molecule designs, where the one or more property computation models are trained to approximate a probability distribution of each property of the plurality of properties;
determining, based at least on the output of the one or more property computation models, a cumulative distribution function (CDF) indicator for each molecule design of the plurality of molecule designs, where the cumulative distribution function (CDF) indicator of a molecule design corresponds to a multivariate rank of the molecule design, and where the multivariate rank of the molecule design quantifies a probability that none of the plurality of properties present in the molecule design can be improved without degrading at least one other property of the plurality of properties; and
selecting, based at least on the cumulative distribution function (CDF) indicator of each molecule design, one or more molecule designs from the plurality of molecule designs as candidates for wet lab assessment.

27. The system of claim 1, wherein the probability distribution of each property of the plurality of properties form a multivariate joint distribution, wherein the determining the cumulative distribution function (CDF) indicator includes decomposing the multivariate joint distribution into one or more bivariate distributions, and wherein each bivariate distribution of the one or more bivariate distributions include a marginal distribution of two or more properties of the plurality of properties and a function coupling the marginal distribution of the two or more properties of the plurality of properties.

28. The system of claim 1, wherein each molecule design of the plurality of molecule designs comprises an antibody.

29. The system of claim 1, wherein the plurality of properties include one or more of expression level, binding affinity, binding specificity, biological activity, and thermostability.

30. The system of claim 1, further comprising:
synthesizing, using one or more lab equipment, the one or more molecule designs selected for wet lab assessment.
